(19) United States
(12) Patent Application Publication
(10) Pub. No.: US 2026/0351342 A1
(43) Pub. Date: Nov. 5, 2026

(54) LINKING HUMAN GENES TO CLINICAL PHENOTYPES USING GRAPH NEURAL NETWORKS

(71) Applicant: Illumina, Inc., San Diego, CA (US)

(72) Inventors: Aashish Nath Adhikari, Oakland, CA (US); Kai-How Farh, Hillsborough, CA (US)

(21) Appl. No.: 19/488,548

(22) PCT Filed: Jun. 2, 2024
(86) PCT No.: PCT/US2024/032170

§ 371 Date: Nov. 26, 2025

Related U.S. Application Data
(60) Provisional application No. 63/505,736, filed on Jun. 2, 2023.

(51) Int. Cl. G16B 20/20; G06F 16/22; G06F 16/28; G16B 40/20; G16B 40/30

(52) U.S. Cl. CPC G16B 20/20; G06F 16/2264; G06F 16/288; G16B 40/20; G16B 40/30

(57) ABSTRACT

This disclosure describes methods, non-transitory computer readable media, and systems that can utilize one or more machine learning models to predict relationships between human genes and phenotypes. For example, the disclosed systems can generate for human genes in relation to various clinical phenotypes. As a basis for generating such predictions, the disclosed systems can train a gene embedding neural network to determine relationships between genes and phenotypes using a two-stage training process that includes a supervised training stage and an unsupervised fine-tuning stage. In addition, the disclosed systems can utilize the gene embedding neural network to generate gene-to-phenotype scores indicating relationships between genes and phenotypes based on similarities among genes (as represented by gene embeddings). Further, the disclosed systems can utilize a diagnostic variant model to determine whether genomic samples exhibit diagnostic variants based on gene-to-phenotype scores as well as other variant-level features of the genomic samples.

### CROSS-REFERENCE TO RELATED APPLICATIONS

This application claims priority to and the benefit of U.S. Provisional Patent Application No. 63/505,736, entitled “LINKING HUMAN GENES TO CLINICAL PHENOTYPES USING GRAPH NEURAL NETWORKS,” filed on Jun. 2, 2023, which is incorporated herein by reference in its entirety.

### BACKGROUND

In recent years, biotechnology firms and computer science institutions have improved hardware and software for generating genetic diagnostics from nucleotide sequences of genomic samples. In particular, some existing diagnostic platforms generate variant calls from nucleotide reads of a genomic sample and/or run diagnostics on variant calls for a variety of purposes. For example, some existing diagnostic systems perform a diagnostic application (e.g., a cancer screening assay) to screen a variant call from a genomic sample for cancer markers by detecting specific genetic markers within nucleotide base calls of reads sequenced from a genomic sample. Such cancer markers are often limited to clinically validated variants for which a consensus of data indicates the variants cause or increase the likelihood of a particular form of cancer. Some existing diagnostic systems perform other diagnostics as well, such as genetic testing for cystic fibrosis, Huntington's disease, von Willebrand disease, and other genetic conditions (or propensities for developing genetic conditions) or for determining other genetic traits.

Despite these recent advances, existing diagnostic systems continue to exhibit a number of drawbacks or disadvantages. For example, many conventional genetic diagnostic systems are limited in their scope and utility because only a subset of human genes have been annotated with their corresponding clinical impact based on a consensus of medical data. Indeed, many conventional systems can only perform genetic diagnostics on genes that have been clinically annotated with corresponding phenotypes. Because approximately 5,000-6,000 genes of the approximately 20,000 human genes have been linked to particular phenotypes in genetic phenotype databases, such as Online Mendelian Inheritance in Man (OMIM) or Orphanet Rare Disease Ontology (ORDO), conventional system cannot link variants or reference calls for most human genes to a particular phenotype. While some existing systems can generate and utilize biological network data to capture relationships among human genes and their homologs beyond clinical annotations (e.g., by quantifying pathogenicity impact of genetic variants), these existing systems nevertheless struggle (or fail entirely) to link the genetic relationships or the measures of pathogenicity to particular diseases (or other phenotypes).

Given the limits of genetic phenotype databases and conventional genetic diagnostic systems, some existing pathogenicity prediction models generate predictions that estimate a degree to which amino-acid variants are benign or pathogenic-but without a target phenotype for measurement of benign-ness or pathogenicity. Such pathogenicity predictions can indicate whether an amino-acid variant is likely to cause some diseases, such as certain cancers, developmental disorders, or heart conditions, but cannot provide predictions for amino-acid variants corresponding to approximately 15,000 human genes.

Due at least in part to their inflexible nature and lack of data concerning genes and phenotype, some conventional genetic diagnostic systems exhibit limitations in application and/or inaccuracies/inabilities in linking pathogenic variants to particular diseases. For example, because some existing systems are limited to determining pathogenicity and/or phenotypes for human genes that have been clinically annotated, some systems inaccurately (or cannot) determine correlations between variant nucleotides in a genomic sample and diseases. Indeed, without determining whether a particular gene impacted by a pathogenic variant is associated with phenotypes observed in organisms (e.g., human patients) with a particular disease or other particular phenotype, existing systems cannot accurately determine relationships between the variant and the particular disease or other phenotype.

### SUMMARY

This disclosure describes embodiments of methods, non-transitory computer readable media, and systems that can utilize one or more machine learning models to predict relationships between human genes and phenotypes. For example, the disclosed systems can generate predictions for human genes in relation to various clinical phenotypes. As a basis for generating such predictions, the disclosed systems can train a gene embedding neural network to determine relationships between genes and phenotypes using a two-stage training process that includes a supervised training stage and an unsupervised fine-tuning stage. In addition, the disclosed systems can utilize the gene embedding neural network to generate gene-to-phenotype scores indicating relationships between genes and phenotypes based on similarities among genes (as represented by gene embeddings). Further, the disclosed systems can utilize a diagnostic variant model to determine whether genomic samples exhibit diagnostic variants based on gene-to-phenotype scores as well as other variant-level features of the genomic samples, such as pathogenicity scores or allele frequencies for variant nucleotides.

### BRIEF DESCRIPTION OF THE DRAWINGS

The detailed description refers to the drawings briefly described below.

FIG. 1 illustrates a block diagram of a sequencing system including a phenotype prediction system in accordance with one or more embodiments.

FIG. 2 illustrates an overview of training a gene embedding neural network using a two-stage training process in accordance with one or more embodiments.

FIG. 3 illustrates an example diagram for learning parameters of a gene embedding neural network in accordance with one or more embodiments.

FIG. 4 illustrates a diagram for fine-tuning network parameters of a gene embedding neural network in accordance with one or more embodiments.

FIG. 5 illustrates an example diagram for generating gene-to-phenotype scores for a gene-to-phenotype matrix and/or for different levels of phenotype granularity in accordance with one or more embodiments.

FIG. 6 illustrates an example overview of generating gene-to-phenotype scores in accordance with one or more embodiments.

FIG. 7 illustrates an example diagram for generating and utilizing gene embeddings and gene-to-phenotype scores in accordance with one or more embodiments.

FIG. 8 illustrates an example graph of results of the phenotype prediction system predicting phenotypes from gene embeddings in accordance with one or more embodiments.

FIG. 9 illustrates an example overview of determining whether a genomic sample includes a diagnostic variant in accordance with one or more embodiments.

FIG. 10 illustrates an example diagram for generating variant-to-phenotype scores using a diagnostic variant model in accordance with one or more embodiments.

FIG. 11 illustrates an example graph of performance of predicting diagnostic variants for the phenotype prediction system in accordance with one or more embodiments.

FIG. 12 illustrates a flowchart of a series of acts for generating gene-to-phenotype scores in accordance with one or more embodiments.

FIG. 13 illustrates a flowchart of a series of acts for generating variant-to-phenotype scores in accordance with one or more embodiments.

FIG. 14 illustrates a block diagram of an example computing device for implementing one or more embodiments of the present disclosure.

### DETAILED DESCRIPTION

This disclosure describes embodiments of a phenotype prediction system that generates scores that indicate a link between genes and phenotypes using various machine learning models. To elaborate, the phenotype prediction system links human genes to clinical phenotypes by determining relationships in a graph neural network between genes such that those genes with clinically annotated phenotypes inform or influence the probability of neighboring (or otherwise related) genes (e.g., genes without corresponding clinically annotated phenotypes) also being associated with (or impacting or influencing) the same phenotypes within the graph neural network. For example, the phenotype prediction system generates gene embeddings encoding the relationships between genes in a latent space and further generates gene-to-phenotype scores linking genes to phenotypes (e.g., selected from a limited set of annotated phenotypes) based on the gene embeddings. In addition, the phenotype prediction system leverages the gene-to-phenotype scores (in addition to other features) within a downstream diagnostic variant model to generate variant-to-phenotype scores indicating probabilities of gene variants corresponding to clinical phenotypes. Accordingly, the phenotype prediction system can link human genes to particular phenotypes for determining diagnostic variants from genomic samples.

As just mentioned, in certain implementations, the phenotype prediction system can link human genes to specific phenotypes using various machine learning techniques. To achieve these predictions, the phenotype prediction system generates or trains the machine learning models for specific tasks. For instance, the phenotype prediction system trains a gene embedding neural network to generate gene embeddings and gene-to-phenotype scores. In some embodiments, for instance, the phenotype prediction system utilizes a two-stage training process that includes: 1) an unsupervised training stage that learns the structure of gene graphs for generating gene embeddings defining or encoding relationships between genes, and 2) a supervised fine-tuning stage that incorporates a set of known phenotype labels for tuning network parameters to generate gene-to-phenotype scores indicating strengths of relationships between genes and phenotypes (e.g., probabilities of genes being associated with phenotypes).

Based on training a gene embedding neural network, in some embodiments, the phenotype prediction system generates gene-to-phenotype scores for genes of a genome (e.g., reference genes represented by a reference human genome). For example, the phenotype prediction system identifies genes of a human genome and inputs a set of gene-level (or variant-level) features for the genes (along with names for the genes) into a trained gene embedding neural network. For example, the phenotype prediction system determines whether a particular gene exists within (or belongs to) various biological gene sets within a particular database and indicates (e.g., with a Boolean value of 0 or 1), as gene-level features, membership in each set of a number of sets. In some cases, the phenotype prediction system uses the MSigDB that includes n different gene sets to generate a set of n gene-level features for each gene (with 0 or 1 values to indicate membership in each of the sets).

In turn, the gene embedding neural network, using its parameters learned and tuned via the two-stage training process, generates gene-to-phenotype scores indicating probabilities of the genes being associated with various phenotypes. Indeed, the phenotype prediction system can utilize the gene embedding neural network to generate, for each gene of a human genome, a set of gene-to-phenotype scores where each score indicates a probability of a gene being associated with (e.g., being linked to, impacting, or affecting the expression of) a certain phenotype. To generate the gene-to-phenotype scores, the gene embedding neural network generates gene embeddings encoding relationships between genes such that the gene embedding neural network can leverage the encoded relationships of a gene having clinically annotated phenotype labels for determining probabilities that other, related genes also correspond to the same clinically annotated phenotypes.

As further mentioned, in certain implementations, the phenotype prediction system can utilize gene-to-phenotype scores to determine a diagnostic variant for an organism (e.g., a human) or a genomic sample. For example, the phenotype prediction system can utilize a diagnostic variant model to process gene-to-phenotype scores and variant-level features associated with one or more variant nucleotides of a genomic sample. The diagnostic variant model can accordingly generate variant-to-phenotype scores indicating probabilities of variant nucleotides from a particular genomic sample being associated with (or corresponding to or being linked to or impacting or affecting the expression of) phenotypes determined or identified for the organism (or for the genomic sample). Indeed, the phenotype prediction system can utilize the diagnostic variant model to link variants of a particular human patient to phenotypes observed in the patient to indicate (probabilities of) which variants are affecting the expression of which observed phenotypes (e.g., via the variant-to-phenotype scores).

As suggested above, the phenotype prediction system provide several advantages, benefits, and/or improvements over existing diagnostic systems. For instance, the phenotype prediction system introduces a first-of-its-kind gene embedding neural network that performs new functions not available in prior systems by generating gene embeddings that bridge the gap between genes with annotated phenotypes and genes without annotated phenotypes. Indeed, while some existing systems can only perform genomic diagnostics on genes that have been clinically annotated with phenotype labels, the phenotype prediction system can generate predictions in the form of gene embeddings that encode relationships between un-annotated genes and phenotypes. By predicting and encoding relationships between genes using gene embeddings, the phenotype prediction system can further use these encoded relationships as a basis to predict and encode relationships between genes and phenotype labels. For example, a first set of genes with strong relationships with a second set of genes known to impact phenotypes (as encoded by proximity of gene embeddings) may also impact those same phenotypes, even when the first set of genes lack clinically annotated phenotype labels or other similar phenotype information. Accordingly, the phenotype prediction system can encode relationships between genes and phenotypes using gene embeddings and gene-to-phenotype scores to locate or identify genes that explain or correlate with respective phenotype labels (e.g., rare diseases). Indeed, beyond using biological network data to capture relationships among genes, the phenotype prediction system can link genes to particular diseases or other phenotypes using gene embeddings output by a gene embedding neural network, gene-to-phenotype scores output by the gene embedding neural network or processed by a diagnostic variant model, and/or variant-to-phenotype scores output by the diagnostic variant model.

While existing systems struggle to capture or represent biological functions that result from an interplay between a network of genes (and other biomolecules), the phenotype prediction system can generate gene-to-phenotype scores and/or variant-to-phenotype scores that capture this interplay, indicating relationships between genes and phenotypes, even for genes without clinical annotations. Whereas existing genetic diagnostic systems are limited to relationships of approximately 5,000-6,000 genes, the disclosed gene embedding neural network can expand data to link approximately 20,000 human genes to phenotypes. Accordingly, the phenotype prediction system can thus improve flexibility over prior systems by adapting to predict phenotypes for genes that do not have corresponding clinically annotated phenotype labels.

To accomplish the aforementioned improved diagnostic variant predictions, as indicated, the phenotype prediction system utilizes an improved and unique machine-learning model-the gene embedding neural network-that is trained to perform new applications. Unlike existing systems that operate on a limited set of clinically annotated genes from human genomes, the phenotype prediction system uses the gene embedding neural network to generate predicted relationships between un-annotated genes and phenotypes. In some embodiments, the phenotype prediction system further generates or trains the gene embedding neural network using a unique two-stage training process that includes an unsupervised training stage to learn genetic relationships and a supervised fine-tuning to link the genetic relationships to phenotypes.

In addition to newly linked relationships, the phenotype prediction system can link certain pathogenic variants to particular phenotypes (e.g., diseases) or expand target genes for a panel test or gene array that evaluates possible genetic causes or links of a phenotype. To illustrate the latter application, in some cases, given an existing list of genes for a panel or array of genes for sequencing and genotype calling from a genomic sample, the phenotype prediction system can expand the panel by identifying new genes that are in close proximity to the existing panel genes in the gene embedding space. This type of gene embedding leverages the notion of a phenotype cluster of gene embeddings where genes associated with the same phenotype tend to be closer to each other in the gene embedding space. As opposed to prior systems that cannot either determine or determine accurately correlations between variant nucleotides (e.g., pathogenic variants) and diseases or other phenotypes, the phenotype prediction system can accurately determine relationships between pathogenic variant and particular diseases. Indeed, such determinations are possible as a consequence of determining gene embeddings indicating whether a particular gene impacted by (or otherwise associated with) a pathogenic variant is associated with phenotypes observed in an organism, such as a human patient (e.g., using a gene embedding neural network). By leveraging gene embeddings output by a gene embedding neural network, the phenotype prediction system can also identify genes nearby each other in latent space that are likely to be associated with a phenotype and identify nearby genes relevant to the phenotype to a gene panel or array for testing genomic samples for potential causes of (or other links to) the phenotype.

As suggested by the foregoing discussion, this disclosure utilizes a variety of terms to describe features and benefits of the phenotype prediction system. Additional detail is hereafter provided regarding the meaning of these terms as used in this disclosure. As used herein, the term “genomic sample” refers to a sequence of one or more nucleotides generated from, or observed within, a genome of an organism (e.g., a human). For example, a genomic sample includes one or more sequences of nucleotides isolated or extracted from a sample organism (or a copy of such an isolated or extracted sequence). In particular, in some embodiments, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device.

As further used herein, the term “variant nucleotide” (or sometimes simply “variant”) refers to a nucleotide within a sequence that varies from a reference nucleotide at a corresponding genomic coordinate. For example, a variant nucleotide includes a variation (e.g., deletion, insertion, translocation, inversion, or some other variation) in an organism's chromosome or a variation to the nucleotide sequences of the organism's chromosome. Relatedly, as used herein, the term “diagnostic variant” refers to a variant nucleotide that is diagnosed as corresponding to, or impacting the expression of, a particular phenotype. For example, a diagnostic variant includes a variant that causes or affects the expression of a certain genetic condition or disease within an organism.

As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequenced determined by scientists as representative of an organism of a particular species. For example, a linear human reference genome may be GRCh38 or other versions of reference genomes from the Genome Reference Consortium. As a further example, a reference genome may include a reference graph genome that includes both a linear reference genome and paths representing nucleic acid sequences from ancestral haplotypes, such as Illumina DRAGEN Graph Reference Genome hg19.

In addition, as used herein, the term “gene embedding” refers to a representation of a gene in a graphical space as generated, encoded, or extracted from a gene-to-gene graph. For example, a gene embedding includes a latent vector representation of a portion of a gene-to-gene graph. In some cases, a gene embedding includes a latent vector encoding features of a gene and its relationship to other genes from a gene-to-gene graph. For instance, a gene embedding includes or refers to an encoding of gene-level features, including or in addition to a gene's name, from a feature vector, feature tensor, or other feature representation. In some cases, a gene embedding comprises a concatenation of one or more Boolean values indicating membership of an embedded gene within respective gene sets/groupings (e.g., where 0 indicates non-membership in a set/group and 1 indicates membership in the set/group), such as the gene sets in the MSigDB database. Alternatively, in some embodiments, a gene embedding is derived or generated from a concatenation of one or more Boolean values indicating membership of an embedded gene within respective gene sets/groupings (e.g., where 0 indicates non-membership in a set/group and 1 indicates membership in the set/group), such as the gene sets in the MSigDB database. Relatedly, a “gene-to-gene graph” refers to a graph that maps genes (e.g., as nodes) to other genes using edges between gene nodes. In some cases, a gene-to-gene graph is multidimensional (e.g., three-dimensional), such as a gene co-expression graph (Gtex), a protein-protein interaction graph (String), a sequence similarity graph, or a genetic interactions graph (CRISPRi gene pairs).

Relatedly, the term “phenotype cluster” refers to a grouping of gene embeddings associated with a phenotype. For instance, a phenotype cluster includes a grouping of latent vectors representing genes in latent space that cause, influence, or are otherwise associated with a phenotype. In some instances, a phenotype cluster includes gene embeddings within a threshold distance from a centroid (e.g., a center or target gene embedding) or from one another within latent space. As explained below, in some cases, the phenotype prediction system identifies genes representing by gene embeddings in a phenotype cluster that can form a (or add to an existing) list of genes for a gene panel or gene array that assays extracted nucleotides from a genomic sample for variants associated with one or more phenotypes.

As suggested above, the phenotype prediction system can utilize one or more machine learning models to map variants of an organism to phenotypes exhibited by the organism. As used herein, the term “machine-learning model” refers to a computer algorithm or a collection of computer algorithms that automatically improve for a particular task through experience based on use of data. For example, a machine-learning model can utilize one or more learning techniques to improve in accuracy and/or effectiveness. Example machine-learning models include various types of decision trees, logistic regressions, linear regressions, random forests, support vector machines, Bayesian networks, or neural networks.

Relatedly, as used herein, the term “diagnostic variant model” refers to a machine-learning model that determines links or relationships between nucleotide variants and observed phenotypes. For example, a diagnostic variant model includes a logistic regression model (or some other model architecture) that processes gene-to-phenotype scores and/or variant-level features to generate variant-to-phenotype scores indicating probabilities of variants of a sample/organism being associated with (affecting expression of) phenotypes exhibited by the sample/organism. Relatedly, the term “variant-to-phenotype score” refers to a score or a metric that represents or defines a relationship between a variant nucleotide and a phenotype. For example, a variant-to-phenotype score indicates a probability of a particular nucleotide variant of an organism corresponding to, being associated with, or causing the expression of a phenotype (e.g., a disease) observed within or exhibited by the organism.

In addition, as used herein, the term “variant-level feature” refers to a feature or a metric (or some other data) that measures, quantifies, or compares a nucleotide variant with respect to other nucleotide variants or reference nucleotides. For example, a variant-level feature includes a pathogenicity score (e.g., from a PrimateAI model) indicating a degree to which variant proteins corresponding to variant nucleotides are benign or pathogenic, a splice-site score (e.g., from a SpliceAI model) indicating a probability that the variant nucleotides are part of a splice site or a non-splice site for pre-messenger RNA, an allele frequency corresponding to the variant nucleotides (e.g., from a gnomADg_AF model), a genotype for genes of a genomic sample, a loss-of-function status indicating that variant proteins reduce or destroy protein function, or a de novo status of the variant nucleotides as private variants.

In some embodiments, the phenotype prediction system generates variant-to-phenotype scores from a variant call file associated with a genomic sample. As used herein, the term “variant call file” refers to a digital file that indicates or represents genomic data, such as nucleotide variants (e.g., variant calls) and other data corresponding to nucleotide variants. For example, a variant call format (VCF) file refers to a text file format that contains information about variants at specific genomic coordinates, including meta-information lines, a header line, and data lines where each data line contains information about a single nucleotide (e.g., a single variant) or multiple variants.

As mentioned, in some embodiments, the phenotype prediction system utilizes a machine learning model in the form of a gene embedding neural network to generate gene-to-phenotype scores. As used herein, the term the term “neural network” refers to a machine-learning model that can be trained and/or tuned based on inputs to determine classifications or approximate unknown functions. For example, a neural network includes a model of interconnected artificial neurons (e.g., organized in layers) that communicate and learn to approximate complex functions and generate outputs (e.g., gene embeddings and/or gene-to-phenotype scores) based on a plurality of inputs provided to the neural network. In some cases, a neural network refers to an algorithm (or set of algorithms) that implements deep learning techniques to model high-level abstractions in data. For example, a neural network can include a convolutional neural network, a recurrent neural network (e.g., an LSTM), a graph neural network, a self-attention transformer neural network, or a generative adversarial neural network.

Along these lines, the term “gene embedding neural network” refers to a neural network that generates gene embeddings and/or gene-to-phenotype scores. For example, a gene embedding neural network includes a graph neural network architecture with parameters trained using a two-stage training process to encode genetic relationships in gene embeddings and to further incorporate phenotype labels to predict associations or correspondences of encoded genes to the phenotype labels (e.g., in the form of gene-to-phenotype scores). Indeed a “gene-to-phenotype score” refers to a score that indicates or reflects a probability of a gene corresponding to, being associated with, or causing the expression of a phenotype. For instance, a gene-to-phenotype score indicates a strength of a relationship between a given gene and a given phenotype, such as a rare disease or some other clinically annotated phenotype (e.g., a phenotype with a corresponding clinical label as determined by a clinician and/or by a consensus of systems and/or experts).

In some cases, the phenotype prediction system generates a “gene-to-phenotype matrix” from gene-to-phenotype scores, where the gene-to-phenotype matrix maps genes to phenotypes by indicating (e.g., using numbers or other visual markers), for each of a set of genes, gene-to-phenotype score for each of a set of phenotypes. In some cases, a gene-to-phenotype matrix visually indicates a measure or a degree of correspondence of a gene to a phenotype by a color, a shade, and/or a size of an indicator within the gene-to-phenotype matrix.

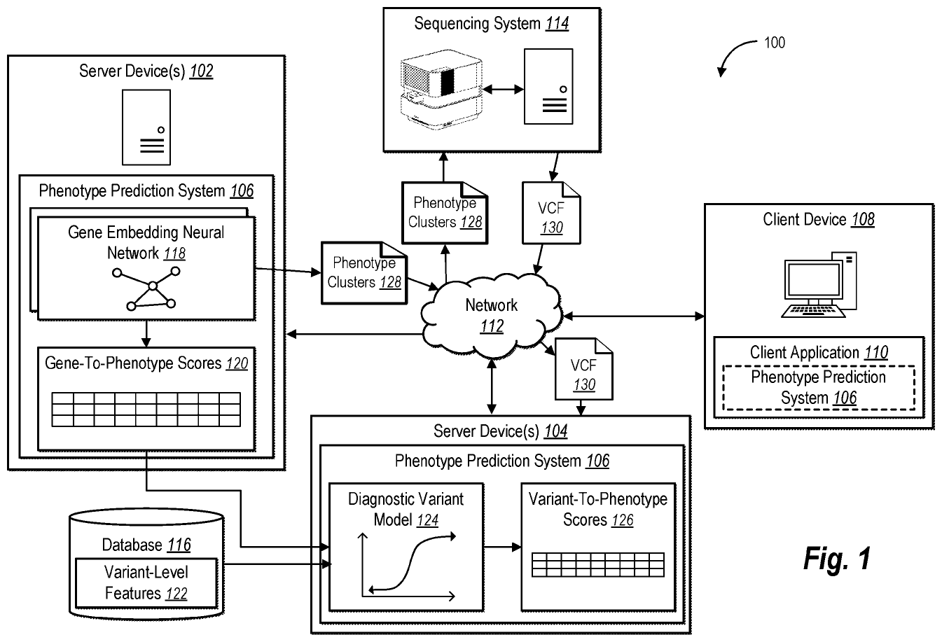

The following paragraphs describe the phenotype prediction system with respect to illustrative figures that portray example embodiments and implementations. For example, FIG. 1 illustrates a schematic diagram of a computing system 100 in which a phenotype prediction system 106 operates in accordance with one or more embodiments. As illustrated, the computing system 100 includes different systems and devices connected via a network 112, including server device(s) 102, server device(s) 104, a client device 108, a database 116, and a sequencing system 114. While FIG. 1 shows an embodiment of the phenotype prediction system 106, this disclosure describes alternative embodiments and configurations below.

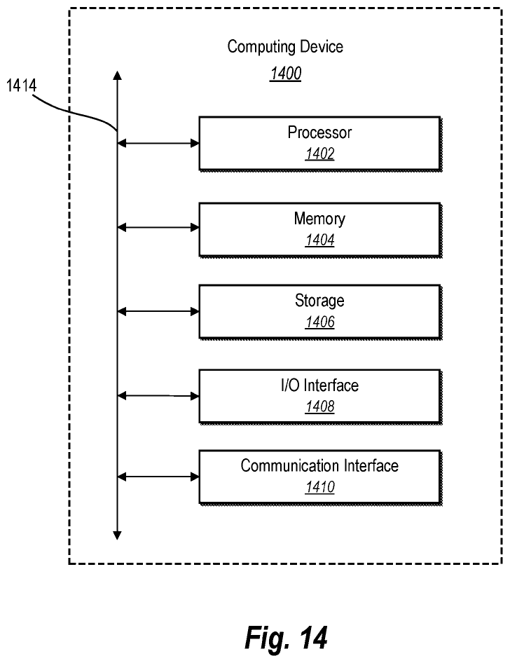

As shown in FIG. 1, the server device(s) 102, the server device(s) 104, the database 116, the client device 108, and the sequencing system 114 can communicate with each other via the network 112. The network 112 comprises any suitable network over which computing devices can communicate. Example networks are discussed in additional detail below with respect to FIG. 14.

As indicated by FIG. 1, the sequencing system 114 comprises a device for sequencing a nucleic acid polymer (e.g., a sequencing device). In some embodiments, the sequencing system 114 uses a sequencing device to analyze nucleic acid segments or oligonucleotides extracted from genomic samples to generate nucleotide reads or other data utilizing computer implemented methods and systems either directly or indirectly on the sequencing device. More particularly, the sequencing system 114 receives and analyzes (e.g., via the sequencing device), within nucleotide-sample slides (e.g., flow cells), nucleic acid sequences extracted from samples. In one or more embodiments, the sequencing system 114 utilizes sequencing-by-synthesis (SBS) to sequence nucleic acid polymers into nucleotide reads. As shown, the sequencing system 114 can receive phenotype clusters 128 (e.g., via the network 112) of gene embeddings representing genes associated with a particular phenotype, identify genes from one or more of the phenotype clusters 128, and either create a list de novo or add to an existing list of genes for a panel or array of genes for sequencing and genotype calling from a genomic sample. Based on such a gene panel or array or a whole-genome sequencing run, the sequencing system 114 can analyze nucleotide reads corresponding to a genomic sample to generate a variant call file 130 (represented by “VCF”). Indeed, the sequencing system 114 can include a computing device, such as a server (e.g., an edge server), to analyze or receive sequencing data from a sequencing device and/or a list of genes corresponding to the phenotype clusters 128 to generate the variant call file 130 comprising variant calls and other nucleotide variant data. In addition or in the alternative to communicating across the network 112, in some embodiments, the sequencing system 114 bypasses the network 112 and communicates directly with the client device 108.

As further indicated by FIG. 1, the server device(s) 102 may generate, receive, analyze, store, and transmit digital data, such as data for generating gene-to-phenotype scores 120, and/or training a gene embedding neural network 118 (e.g., utilizing data from the sequencing system 114 or elsewhere). As shown, the server device(s) 102 can house all or part of the phenotype prediction system 106, including a gene embedding neural network 118. Indeed, using the gene embedding neural network 118, the server device(s) 102 can generate gene-to-phenotype scores 120 and/or phenotype clusters 128 based on genes from a reference genome, where the gene-to-phenotype scores 120 and/or phenotype clusters 128 can be used to determine diagnostic variants in combination with sequencing data (e.g., generated by, and received from, the sequencing system 114). The server device(s) 102 may also communicate with the client device 108 to send data to the client device 108, including information indicating the gene-to-phenotype scores 120, phenotype clusters 128, nucleotide variants, variant-level features (e.g., variant-level features 122), or other data.

As further illustrated in FIG. 1, the computing system 100 can also include server device(s) 104. The server device(s) 104 may generate, receive, store, and transmit digital data, such as data for generating variant-to-phenotype scores 126. For example, the server device(s) 104 may house all or part of the phenotype prediction system 106, including a diagnostic variant model 124 for generating the variant-to-phenotype scores 126 based on variant-level features 122 (e.g., stored within the database 116) and the variant call file 130 (e.g., generated by, and received from, the sequencing system 114). Indeed, as shown in FIG. 1, the sequencing system 114 may send (and the server device(s) 104 may receive) call data in the form of the variant call file 130 or “VCF.” The server device(s) 104 may also communicate with the client device 108. In particular, the server device(s) 104 can send data to the client device 108, including the variant call file 130 or other information indicating nucleotide variants, the variant-level features 122, or other data.

In some embodiments, the server device(s) 102 and/or the server device(s) 104 comprise distributed collections of servers where the server device(s) 102 and/or the server device(s) 104 include a number of server devices distributed across the network 112 and located in the same or different physical locations. Further, the server device(s) 102 and/or the server device(s)104 can each comprise a content server, an application server, a communication server, a web-hosting server, or another type of server. While FIG. 1 depicts the gene embedding neural network 118 and the diagnostic variant model 124 hosted on the server device(s) 102 and the server device(s) 104, respectively, the gene embedding neural network 118 and the diagnostic variant model 124 and other components of the phenotype prediction system 106 can be hosted on a same server device. Accordingly, in some cases, the server device(s) 102 and/or the server device(s) 104 are the same server device(s), are located at the same physical location, and/or share a physical location with the sequencing system 114 and/or the database 116.

As mentioned, and as illustrated in FIG. 1, the phenotype prediction system 106 analyzes data-such as nucleotide variants from the variant call file 130 output by the sequencing system 114 along with gene-to-gene graphs, gene embeddings, and/or variant-level features (e.g., variant-level features 122), and/or other data-to generate gene-to-phenotype scores 120 and/or the variant-to-phenotype scores 126 to link nucleotide variants to phenotypes (e.g., diseases). In some cases, the phenotype prediction system 106 includes a gene embedding neural network 118 and/or a diagnostic variant model 124. Indeed, in some embodiments, the phenotype prediction system 106 trains or generates the gene embedding neural network 118 by learning network parameters according to a particular two-stage training process described further below.

As further illustrated and indicated in FIG. 1, the client device 108 can generate, store, receive, and send digital data. In particular, the client device 108 can receive data representing gene-to-gene graphs, the phenotype clusters 128, gene embeddings, and/or the variant-level features 122. Furthermore, the client device 108 may communicate with the server device(s) 102 to receive the variant call file 130. The client device 108 can accordingly present or display information pertaining to generating the gene-to-phenotype scores 120 and/or the variant-to-phenotype scores 126 within a graphical user interface to a user associated with the client device 108. The client device 108 illustrated in FIG. 1 may comprise various types of client devices. For example, in some embodiments, the client device 108 includes non-mobile devices, such as desktop computers or servers, or other types of client devices. In yet other embodiments, the client device 108 includes mobile devices, such as laptops, tablets, mobile telephones, or smartphones. Additional details regarding the client device 108 are discussed below with respect to FIG. 14.

As further illustrated in FIG. 1, the client device 108 includes a client application 110. The client application 110 may be a web application or a native application stored and executed on the client device 108 (e.g., a mobile application, desktop application). The client application 110 can include instructions that (when executed) cause the client device 108 to receive data from the phenotype prediction system 106 and present, for display at the client device 108, data pertaining to the gene-to-phenotype scores 120, the variant-to-phenotype scores 126, and/or variant diagnostics.

As further illustrated in FIG. 1, in some embodiments, the phenotype prediction system 106 may be located on the client device 108 as part of the client application 110 or on the sequencing system 114 (e.g., downloaded in whole or in part). Accordingly, in some embodiments, the phenotype prediction system 106 is implemented by (e.g., located entirely or in part) on the client device 108. In yet other embodiments, the phenotype prediction system 106 is implemented by one or more other components of the computing system 100, such as the sequencing system 114. In particular, the phenotype prediction system 106 can be implemented in a variety of different ways across the server device(s) 102 and/or the server device(s) 104, the network 112, the client device 108, and the sequencing system 114. For example, the phenotype prediction system 106 can be downloaded from the server device(s) 102 and/or the server device(s) 104 to the client device 108 and/or to the sequencing system 114 where all or part of the functionality of the phenotype prediction system 106 is performed at each respective device within the computing system 100.

As further illustrated in FIG. 1, the computing system 100 includes a database 116. The database 116 can store information, such as variant call files (e.g., the variant call file 130), gene-to-gene graphs, gene embeddings, the variant-level features 122, and/or other data described herein. In some embodiments, the server device(s) 102, the server device(s) 104, the client device 108, and/or the sequencing system 114 communicate with the database 116 (e.g., via the network 112) to store and/or access information. In some cases, the database 116 also stores one or more models, such as the gene embedding neural network 118 and the diagnostic variant model 124.

Though FIG. 1 illustrates the components of the computing system 100 communicating via the network 112, in certain implementations, the components of computing system 100 can also communicate directly with each other, bypassing the network 112. For instance, and as previously mentioned, in some implementations, the client device 108 communicates directly with the sequencing system 114. Additionally, in some embodiments, the client device 108 communicates directly with the phenotype prediction system 106. Moreover, the phenotype prediction system 106 can access one or more databases housed on or accessed by the server device(s) 102 and/or the server device(s) 104 or elsewhere in the computing system 100.

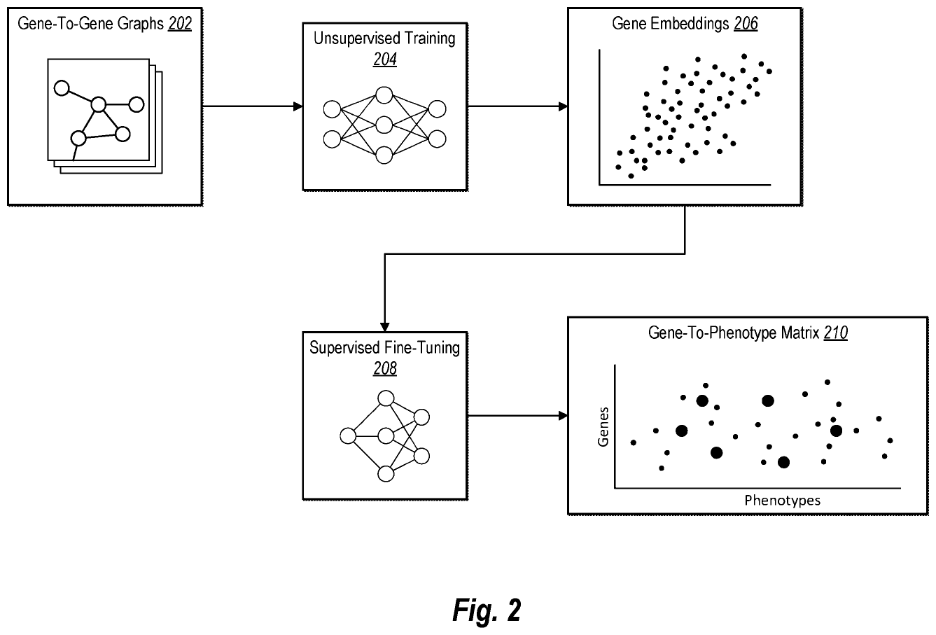

As indicated above, the phenotype prediction system 106 can determine that a nucleotide variant of a genomic sequence for an organism is a diagnostic variant. In particular, the phenotype prediction system 106 can determine links or relationships between nucleotide variants and observed/exhibited phenotypes using the machine learning techniques described herein. As a basis for determining that a variant is diagnostic, the phenotype prediction system 106 can also determine relationships between genes and phenotypes (e.g., in the form of gene-to-phenotype scores) using a gene embedding neural network. For this purpose, the phenotype prediction system 106 can train or build the gene embedding neural network using a two-stage training process. FIG. 2 provides an overview of the two-stage training process for the gene embedding neural network in accordance with one or more embodiments. Additional detail regarding the components illustrated in FIG. 2 is provided thereafter with reference to subsequent figures.

As illustrated in FIG. 2, the phenotype prediction system 106 identifies, receives, or accesses gene-to-gene graphs 202. In particular, the phenotype prediction system 106 accesses gene-to-gene graphs 202 that visually represent relationships between genes. For instance, the phenotype prediction system 106 accesses an external database storing gene-to-gene graphs, such as a gene co-expression graph (Gtex), a protein-protein interaction graph (String), a sequence similarity graph, or a genetic interactions graph (CRISPRi gene pairs).

As further illustrated in FIG. 2, the phenotype prediction system 106 performs unsupervised training 204. More specifically, as the first stage of the two-stage training process for learning parameters of a gene embedding neural network, the phenotype prediction system 106 utilizes an unsupervised process (without ground truth labels) to learn a graph structure of the gene-to-gene graphs 202 (e.g., of one graph or more than one graph). For instance, the phenotype prediction system 106 inputs one or more of the gene-to-gene graphs 202 (or a combination of two or more of the graphs) into a gene embedding neural network. The gene embedding neural network generates a predicted gene embedding, and the phenotype prediction system 106 adjusts network parameters (e.g., weights and biases) based on the predicted gene embedding. The phenotype prediction system 106 repeats the process over training iterations so that eventually the gene embedding neural network learns parameters that accurately model or encode the structure of the input encoding the gene-to-gene graphs 202. Accordingly, the phenotype prediction system 106 generates gene embeddings 206 that represent or encode the genes within a latent space, where the distance between the gene embeddings represents the relationships between the genes (e.g., as indicated by the edges of the gene-to-gene graphs 202).

As further illustrated in FIG. 2, the phenotype prediction system 106 performs a supervised fine-tuning 208 (with ground truth labels for phenotypes) as the second stage within the two-stage training process. To elaborate, the phenotype prediction system 106 fine-tunes the parameters learned from the unsupervised training 204 (e.g., the first stage) to account for phenotype labels. For instance, the phenotype prediction system 106 links genes to phenotypes by encoding or scoring gene-to-phenotype relationships through the supervised fine-tuning. Specifically, the phenotype prediction system 106 tunes network parameters on an attention-based classification task to classify genes into respective phenotype categories or labels. For example, for a gene that has a clinically annotated phenotype label, the phenotype prediction system 106 learns its relationships to nearby genes (e.g., given by the gene embeddings 206) and further links the nearby genes to the phenotype label (e.g., where genes farther from the labeled gene have weaker links than closer genes).

Accordingly, the phenotype prediction system 106 trains the gene embedding neural network to generate a gene-to-phenotype matrix 210 that represents or includes gene-to-phenotype scores. Indeed, for each gene in the matrix, the phenotype prediction system 106 determines a score for each of a set of phenotype labels. As shown, higher gene-to-phenotype scores are represented by larger dots and lower gene-to-phenotypes scores are represented by smaller dots (where some dots are invisible because the scores are so small).

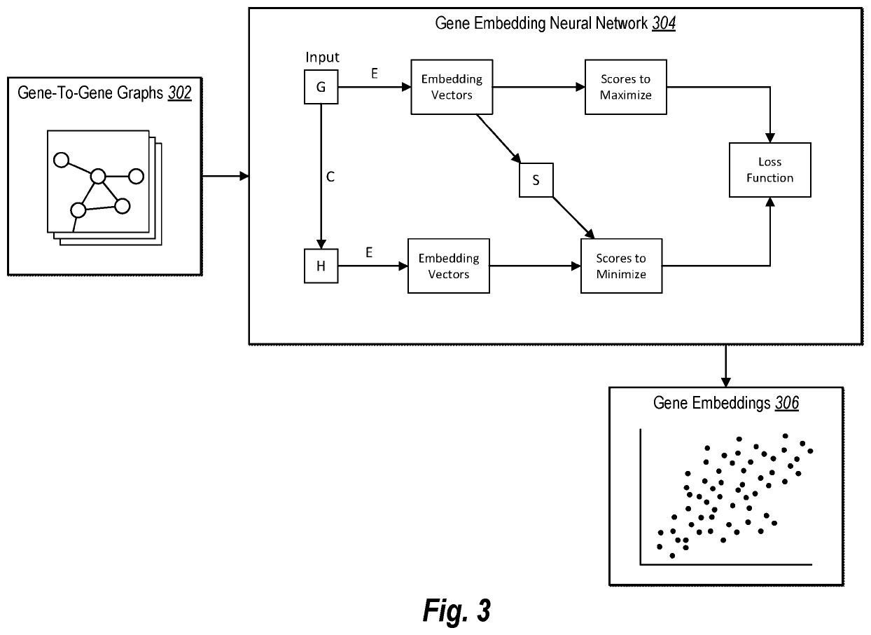

As mentioned, in certain embodiments, the phenotype prediction system 106 utilizes a two-stage training process to train a gene embedding neural network. In particular, the phenotype prediction system 106 utilizes a two-stage training process that includes, as the first stage, an unsupervised training stage. FIG. 3 represents an example diagram for an unsupervised training stage for learning parameters of a gene embedding neural network in accordance with one or more embodiments.

As illustrated in FIG. 3, the phenotype prediction system 106 accesses, receives, or identifies gene-to-gene graphs 302. In particular, the phenotype prediction system 106 accesses a database (e.g., the database 116) that stores gene-to-gene graphs 302. As described, the gene-to-gene graphs 302 model, map, or represent relationships between genes, between proteins, and/or between gene sequences using nodes and edges, where the nodes represent genes (or proteins or sequences), and the edges represent relationships between the nodes.

As further illustrated in FIG. 3, the phenotype prediction system 106 utilizes the gene-to-gene graphs 302 to perform an unsupervised training process to learn parameters of a gene embedding neural network 304. Indeed, the phenotype prediction system 106 learns parameters of the gene embedding neural network 304 such that the parameters extract or encode latent vectors that represent the genes (or the proteins or the gene sequences) and their relationships to other genes as represented by the gene-to-gene graphs 302. For example, the phenotype prediction system 106 learns to encode the structure of the gene-to-gene graphs 302 through the unsupervised training of the gene embedding neural network 304.

As part of the unsupervised training stage, the phenotype prediction system 106 utilizes a graph representation learning algorithm (e.g., a graph attention algorithm or a graph convolution network), represented by E. For example, as part of the algorithm, the phenotype prediction system 106 samples a negative example by using a corruption function. Specifically, the phenotype prediction system 106 corrupts the input encoding the gene-to-gene graphs 302 (represented by G) using the corruption function C to generate corrupted data H. In addition, the phenotype prediction system 106 determines or obtains patch representations for the input G and the corrupted data H (e.g., the negative sample) by utilizing an encoder neural network. Accordingly, the phenotype prediction system 106 generates the patch representations (represented by “Embedding Vectors”).

As further illustrated in FIG. 3 for the gene embedding neural network 304, the phenotype prediction system 106 utilizes a readout function to generate a summarized version of the input G (e.g., the gene-to-gene graphs 302), as represented by S. The phenotype prediction system 106 further determines score to maximize from the embedding vectors of G and scores to minimize from the embedding vectors of H and utilizes a loss function (e.g., a gradient descent loss function) to determine a measure of loss associated with the gene embedding neural network.

The phenotype prediction system 106 further repeats the above-mentioned steps of the graph representation learning algorithm for multiple iterations. Specifically, the phenotype prediction system 106 repeats the graph representation learning algorithm until the gene embedding neural network 304 satisfies a threshold measure of loss and/or achieves a threshold measure of accuracy. For example, the phenotype prediction system 106 applies a graph-attention encoder as part of the graph representation learning algorithm, as described by Petar Veličković, Guillem Cucurull, Arantxa Casanova, Adriana Romero, Pietro Liò, and Yoshua Bengio, “Graph Attention Networks,” Int'l Conf. Learning Representations (2018) (hereinafter Graph Attention Networks), available at arXiv: 1710.10903, which is hereby incorporated by reference in its entirety. In some cases, the phenotype prediction system 106 repeats the algorithm for a threshold number of iterations and/or until maximizing a particular objective function. In these or other cases, the phenotype prediction system 106 utilizes one or more aspects of the graph representation learning algorithm described by Petar Veličković, William Fedus, William L. Hamilton, Pietro Liò, Yoshua Bengio, and R. Devon Hjelm, “Deep Graph Infomax,” Int'l Conf. Learning Representations (2018), available at arXiv: 1809.10341, which is hereby incorporated by reference in its entirety.

Based on this unsupervised training, the phenotype prediction system 106 trains the gene embedding neural network 304 to generate gene embeddings 306. In particular, phenotype prediction system 106 adjusts network parameters until the gene embedding neural network 304 generates the gene embeddings 306 as latent vector representations of genes within a latent space. For instance, the phenotype prediction system 106 generates the gene embeddings 306 in the form of sequences of binary indicators or Boolean values indicating whether genes belong to particular gene sets or gene groups. In some embodiments, the gene embedding neural network 304 predicts whether a gene belongs to a known gene set (e.g., from among the gene sets of MSigDB) and encodes the gene's membership with a 0 (non-member) or a 1 (member). The gene embedding neural network 304 thus generates a number of binary values, one for each known gene set, to generate a gene embedding having a sequence of 0s and 1s indicating membership in the respective sets. Alternatively, in some embodiments, the phenotype prediction system 106 generates the gene embeddings 306 based on sequences of binary indicators or Boolean values indicating whether genes belong to particular gene sets or gene groups, including, but not limited to, the example of binary values just described above.

In some cases, the phenotype prediction system 106 further determines relationships between genes (or between the gene embeddings 306) based on distances between the gene embeddings 306 with the latent space. In some cases, the phenotype prediction system 106 generates the gene embeddings 306 to have a number of dimensions equal to a number of neurons within a penultimate layer of the gene embedding neural network 304. As shown, each dot represents a gene within a two-dimensional latent space, though this is merely an example for illustrative purposes.

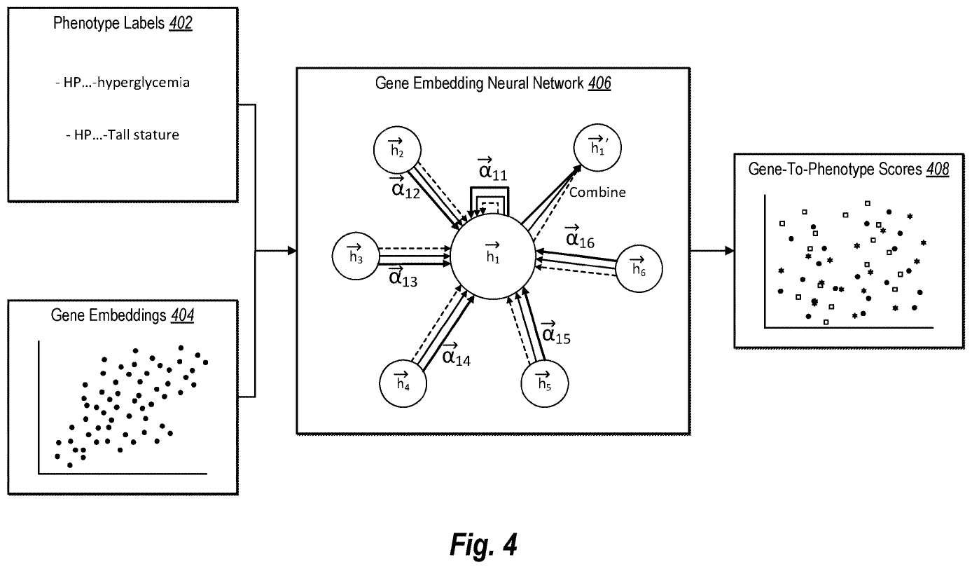

As mentioned, in certain embodiments, the phenotype prediction system 106 performs a fine-tuning process as a second stage within a two-stage training process for a gene embedding neural network. In particular, the phenotype prediction system 106 fine-tunes parameters of a gene embedding learned during the unsupervised first stage described above. FIG. 4 illustrates an example diagram for performing the second, supervised fine-tuning stage for training a gene embedding neural network in accordance with one or more embodiments.

As illustrated in FIG. 4, the phenotype prediction system 106 determines, identifies, receives, or accesses phenotype labels 402. More specifically, the phenotype prediction system 106 accesses or determines labels for a set of clinically annotated phenotypes. For example, the phenotype prediction system 106 accesses a database (e.g., the database 116) storing phenotype labels determined or generated by clinicians, systems, and/or experts for a set of human genes. In some cases, the phenotype prediction system 106 access the phenotype labels from a gene-phenotype database, such as Online Mendelian Inheritance in Man (OMIM) or Orphanet Rare Disease Ontology (ORDO), which include clinical labels for a subset (e.g., five to six thousand) of the approximately twenty thousand genes in a human genome. Indeed, the phenotype labels 402 include labels indicating phenotypes linked to (or affected by) which specific genes. As shown, the phenotype labels 402 include phenotypes for hyperglycemia, tall stature, and others that can be linked to one or more genes of a human genome.

As further illustrated in FIG. 4, the phenotype prediction system 106 accesses, identifies, determines, or generates gene embeddings 404. More particularly, the phenotype prediction system 106 generates the gene embeddings 404 using network parameters learned during the first training stage, as described above. Using the supervised fine-tuning process, the phenotype prediction system 106 tunes the network parameters to learn associations or relationships between the phenotype labels 402 and the gene embeddings 404. Indeed, in some cases, the phenotype prediction system 106 trains the gene embedding neural network 406 (e.g., the gene embedding neural network 304) based on a subset of available phenotype labels of the phenotype labels 402 to learn to associate all the gene embeddings 404 (e.g., for all human genes, including un-annotated genes) with the phenotype labels 402 (e.g., with at least some measure of association or relationship). For example, in some cases, certain gene embeddings closer in latent space to a labeled gene embedding (e.g., a gene embedding corresponding to a gene with a clinically annotated phenotype) are more likely to be associated with the same phenotype as the labeled gene than those farther away in the latent space.

As part of the supervised fine-tuning stage, in some embodiments, the phenotype prediction system 106 utilizes a graph representation learning algorithm (e.g., the same graph representation learning algorithm as for the first training stage or a different variation). To elaborate, the phenotype prediction system 106 utilizes an attention mechanism to link or associate the gene embeddings 404 with the phenotype labels 402. For instance, the phenotype prediction system 106 utilizes an attention mechanism, such as a single-layer feed-forward neural network parameterized by a weight vector, to determine normalized attention coefficients for a given attention head.

As shown in FIG. 4, the gene embedding neural network 406 includes multiple attention heads, represented by \vec{h}₁ through \vec{h}₆, and each of the differently styled arrows represents an independent attention computation. For instance, the phenotype prediction system 106 utilizes softmax functions (represented by \vec{α}_{i, j} where i denotes a node from a gene-to-gene graph and j denotes a measure of importance of node j's features to node i) to determine attention coefficients for each node (or for each gene embedding). In some cases, the phenotype prediction system 106 further combines (e.g., concatenates or averages) the aggregated features from each attention head to determine

$${\vec{h}}_{1}^{′} ,$$

the final output in the form of a gene-to-phenotype score.

In some embodiments, the phenotype prediction system 106 repeats the fine-tuning process for a number of iterations. For instance, the phenotype prediction system 106 repeats the fine-tuning process for a threshold number of iterations and/or until the gene embedding neural network 406 satisfies a threshold measure of loss (e.g., according to a cross-entropy loss function between predicted phenotype labels and ground truth phenotype labels from clinical annotations). Based on the supervised fine-tuning process, the phenotype prediction system 106 utilizes the gene embedding neural network 406 to generate gene-to-phenotype scores 408. Specifically, the phenotype prediction system 106 generates gene-to-phenotype scores 408 that represent or encode relationships between genes (e.g., the gene embeddings 404) and the phenotype labels.

As further indicated by FIG. 4, the phenotype prediction system 106 can further modify the gene embeddings 404 to reflect the gene-to-phenotype scores 408 by grouping or clustering gene embeddings according to corresponding phenotypes (e.g., where the differently shaped gene embedding symbols correspond to different phenotypes). In some cases, the phenotype prediction system 106 trains the gene embedding neural network 304 using the graph representation learning algorithm described by Graph Attention Networks, as incorporated by reference above.

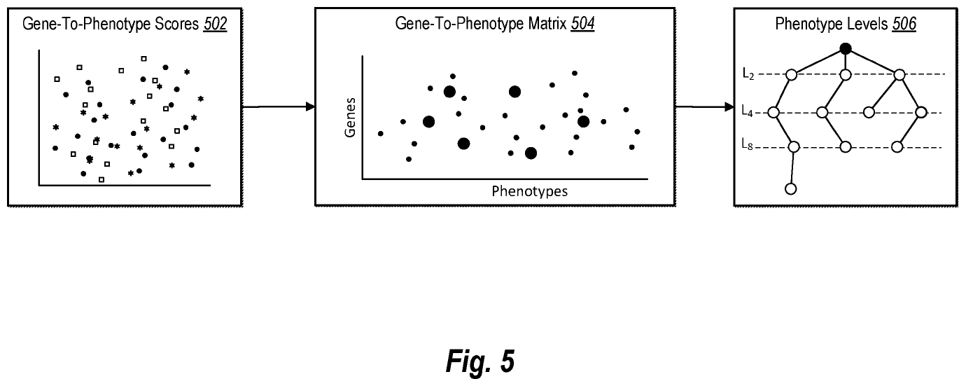

As mentioned, in certain embodiments, the phenotype prediction system 106 utilizes a gene embedding neural network to generate gene-to-phenotype scores. In some cases, the phenotype prediction system 106 represents gene-to-phenotype scores within a gene-to-phenotype matrix. In these or other cases, the phenotype prediction system 106 determines different levels or different granularities of gene-to-phenotype scores. FIG. 5 illustrates an example diagram of different representations of gene-to-phenotype scores in accordance with one or more embodiments.

As illustrated in FIG. 5, the phenotype prediction system 106 determines gene-to-phenotype scores 502. Indeed, as described herein, the phenotype prediction system 106 determines the gene-to-phenotype scores 502 utilizing a gene embedding neural network to encode relationships between genes and labeled phenotypes. As shown, the phenotype prediction system 106 learns how to encode gene embeddings based on relatedness to phenotype labels. In some cases, the phenotype prediction system 106 can further group or cluster gene embeddings based on phenotype scores to, for example, generate phenotype clusters of gene embeddings that most closely correspond to respective phenotypes. As shown, the phenotype prediction system 106 can group gene embeddings on a phenotype-by-phenotype basis, represented by the different shapes which correspond to respective phenotype labels.

As further illustrated in FIG. 5, the phenotype prediction system 106 can generate a gene-to-phenotype matrix 504. In particular, the phenotype prediction system 106 can generate a gene-to-phenotype matrix 504 that indicates relatedness between genes and phenotypes (as determined by the gene-to-phenotype scores 502). For instance, the phenotype prediction system 106 generates a gene-to-phenotype matrix 504 that includes a set of genes (e.g., all genes in a human genome or a genome of another organism) on one axis and a set of phenotype labels on another axis. For each gene, the phenotype prediction system 106 determines a gene-to-phenotype score for each of a set of phenotype labels, where some scores are higher than others (indicating a stronger relationship). Thus, the phenotype prediction system 106 can predict relationships between all genes of a genome and all phenotypes of a set of phenotypes. As shown, the phenotype prediction system 106 generates a gene-to-phenotype matrix 504 where larger dots represent higher gene-to-phenotype scores.

Additionally, in some embodiments, the phenotype prediction system 106 determines or generate phenotype levels 506. In particular, the phenotype prediction system 106 determines levels of phenotypes, where higher-level phenotypes are more general and include multiple sub-categories of phenotypes. For instance, the phenotype prediction system 106 determines a phenotype label for “height abnormality” at a certain phenotype level and further determines phenotype labels for “tall stature” and “short stature” at lower phenotype levels within the height-abnormality label. As shown, the phenotype prediction system 106 can generate one or more phenotype trees that represent phenotype levels. In some cases, the phenotype prediction system 106 utilizes a Human Phenotype Ontology from Genomics England to generate or determine the phenotype levels 506.

In addition, the phenotype prediction system 106 can further determine gene-to-phenotype scores 502 and/or generate a gene-to-phenotype matrix 504 that represents one or more layers of granularity according to the phenotype levels 506. For instance, the phenotype prediction system 106 can generate gene-to-phenotype scores indicating relationships between genes and phenotype categories (e.g., metabolic disease or cardiovascular disease) and/or can generate gene-to-phenotype scores indicating relationships between genes and specific phenotypes (e.g., hypertrophic cardiomyopathy or ketonic hypoglycemia).

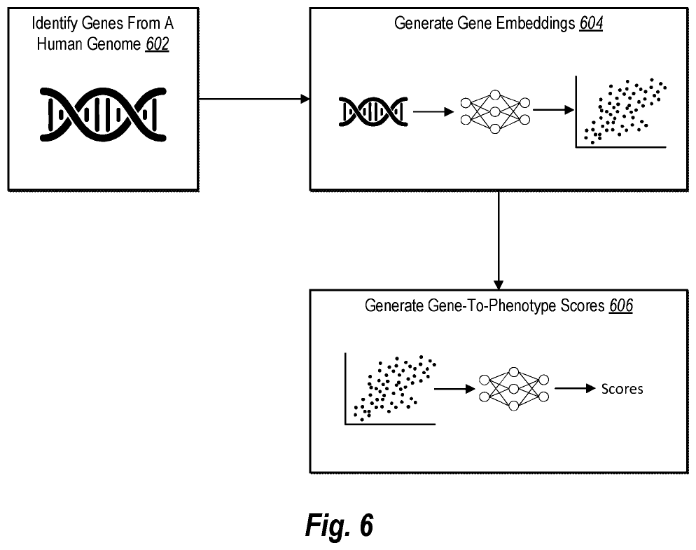

As mentioned, in one or more embodiments, the phenotype prediction system 106 utilizes a trained gene embedding neural network to generate gene-to-phenotype scores for a particular organism. In particular, the phenotype prediction system 106 generates gene-to-phenotype scores for a set of genes of a human genome. FIG. 6 illustrates an example overview of generating gene-to-phenotype scores for a set of genes in accordance with one or more embodiments.

As illustrated in FIG. 6, the phenotype prediction system 106 performs an act 602 to identify genes from a human genome. More specifically, the phenotype prediction system 106 identifies, receives, or accesses a set of genes from a database (or from a sequencing device), such as a reference genome (e.g., a reference human genome) or a genome sequenced from a sample for a human (or some other organism). For instance, the phenotype prediction system 106 identifies or accesses nucleotide sequences representing all genes within a reference genome (e.g., approximately 20,000 genes from a human reference genome). In some cases, the phenotype prediction system 106 identifies all genes for a human genome, while in other cases the phenotype prediction system 106 identifies a subset of genes for a human genome.

In addition, the phenotype prediction system 106 performs an act 604 to generate gene embeddings. Particularly, as described herein, the phenotype prediction system 106 utilizes a gene embedding neural network to generate gene embeddings for the set of genes. Indeed, the phenotype prediction system 106 utilizes a gene embedding neural network to process the set of genes (e.g., in the form of a VCF file or some other computer data representation of the set of genes) to encode the genes into latent vector representations. In some cases, the phenotype prediction system 106 generate the gene embeddings to have a number of dimensions (e.g., a dimensionality) equal to a number of neurons (e.g., 32 neurons for 32 dimensions) within a penultimate layer of a gene embedding neural network.

As further shown in FIG. 6, the phenotype prediction system 106 further performs an act 606 to generate gene-to-phenotype scores. More specifically, the phenotype prediction system 106 utilizes a gene embedding neural network to process the gene embeddings to determine relationships between the gene embeddings and phenotype labels. Indeed, as described, the phenotype prediction system 106 generates the gene-to-phenotype scores using learned parameters of a gene embedding neural network trained on genes and clinically annotated phenotype labels. For example, the phenotype prediction system 106 utilizes an output layer (e.g., a softmax layer) of the gene embedding neural network that affiliates or associates gene embeddings and phenotype layers according to the learned network parameters.

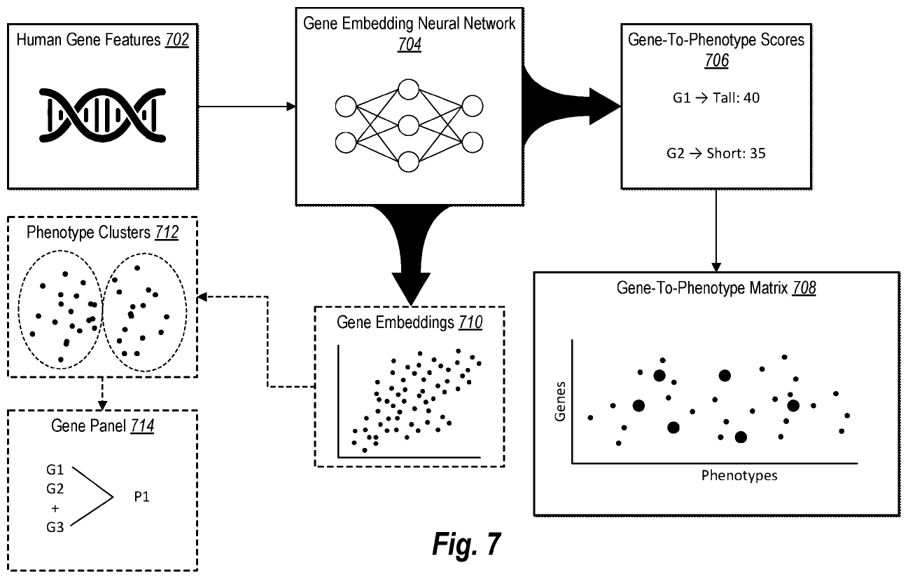

As mentioned above, in certain described embodiments, the phenotype prediction system 106 utilizes gene embeddings and/or gene-to-phenotype scores for one or more genomic analyses. In particular, the phenotype prediction system 106 utilizes gene embeddings to modify, update, or expand gene panels that list genes corresponding to particular phenotypes (e.g., diseases). As another example, the phenotype prediction system 106 utilizes gene-to-phenotype scores to generate a gene-to-phenotype matrix that can be used to predict diagnostic variants in human patients. FIG. 7 illustrates an example process of using a gene embedding neural network to generate gene embeddings and gene-to-phenotype scores for updating gene panels and/or for generating a gene-to-phenotype matrix in accordance with one or more embodiments.

As illustrated in FIG. 7, the phenotype prediction system 106 inputs human genes 702 into a gene embedding neural network 704. As indicated above, in some cases, the phenotype prediction system 106 inputs the human genes 702 in the form of gene-level features (e.g., sequences of binary indicators or Boolean values indicating whether genes belong to particular gene sets or gene groups) into the gene embedding neural network 704. Indeed, as mentioned, the phenotype prediction system 106 inputs a set of genes (or gene-level features) from a human genome, where the set includes all of the genes or a subset of genes from a human genome. In turn, the gene embedding neural network 704 generates gene-to-phenotype scores 706 using network parameters according to the training described herein. As shown, the phenotype prediction system 106 generates gene-to-phenotype scores for gene G1 and gene G2. For example, the phenotype prediction system 106 determines a score of 40 (or some other score on a particular gene-to-phenotype scale) for the relationship between G1 gene and a phenotype of “Tall.” Additionally, the phenotype prediction system 106 determines a score of 35 for the relationship between the G2 gene and a phenotype of “Short.” Likewise, the phenotype prediction system 106 determines other gene-to-phenotype scores for each of G1 and G2 and for others of the human genes 702 as well.

As further shown in FIG. 7, the phenotype prediction system 106 further generates a gene-to-phenotype matrix 708 to encode or represent relationships between genes and phenotypes in matrix form. In particular, the phenotype prediction system 106 represents the gene-to-phenotype scores 706 within a gene-to-phenotype matrix 708 that numerically, graphically, mathematically, and/or visually represents the relationships between each of the human genes 702 and each of a set of phenotype labels.

As further illustrated in FIG. 7, in one or more embodiments, the phenotype prediction system 106 generates gene embeddings 710 utilizing the gene embedding neural network 704. More specifically, the phenotype prediction system 106 generates latent vector representations of the human genes 702 and their relationship to one another according to learned parameters of the gene embedding neural network 704.

In addition, in some embodiments, the phenotype prediction system 106 generates phenotype clusters 712 from the gene embeddings 710. More particularly, the phenotype prediction system 106 determines distances between the gene embeddings 710 as a basis for utilizing a clustering technique (e.g., a k-means clustering algorithm) to cluster the gene embeddings 710 into the phenotype clusters 712 (e.g., such that embeddings within a threshold distance of one another are in a common phenotype-specific cluster). In some cases, the phenotype prediction system 106 incorporates phenotype information as part of generating the phenotype clusters 712. To elaborate, because the gene embedding neural network 704 is trained on genes and phenotype labels, the gene embedding neural network 704 generates the gene embeddings 710 (and the corresponding phenotype clusters 712) that capture gene-phenotype relationships as well as gene-gene relationships. Accordingly, the phenotype prediction system 106 can cluster the gene embeddings 710 into the phenotype clusters 712 such that each cluster represents a respective phenotype (e.g., a respective disease).

As further illustrated in FIG. 7, the phenotype prediction system 106 modifies or updates a gene panel 714 that can be used as part of gene array for sequencing a genomic sample. To elaborate, the phenotype prediction system 106 accesses or receives the gene panel 714 that includes a list of genes associated with a particular phenotype (e.g., a particular disease). For example, the phenotype prediction system 106 receives or accesses (from a database) gene panels that have been created by clinicians, systems, and/or experts to include lists of genes that correspond to (e.g., influence or affect the expression of) a certain phenotype. In addition, the phenotype prediction system 106 updates or modifies the gene panel 714 to add additional genes.

Specifically, the phenotype prediction system 106 identifies a phenotype cluster (e.g., from the phenotype clusters 712) corresponding to the gene panel 714. For example, the phenotype prediction system 106 identifies a phenotype cluster associated with the same phenotype for the gene panel 714. The phenotype prediction system 106 can further add one or more additional genes from the corresponding phenotype cluster to the gene panel 714 or form the gene panel 714 de novo based on genes represented by gene embeddings within a specific phenotype cluster. In some cases, the phenotype prediction system 106 selects one or more genes from the phenotype cluster to add to the gene panel 714, such as genes with gene-to-phenotype scores in relation to the respective phenotype that satisfy a score threshold (or a number of top-scoring genes from the phenotype cluster). As shown, the phenotype prediction system 106 adds gene G3 to the gene panel 714 that already includes genes G1 and G2 based on determining that the gene-to-phenotype score for G3 in relation to phenotype P1 satisfies a score threshold.

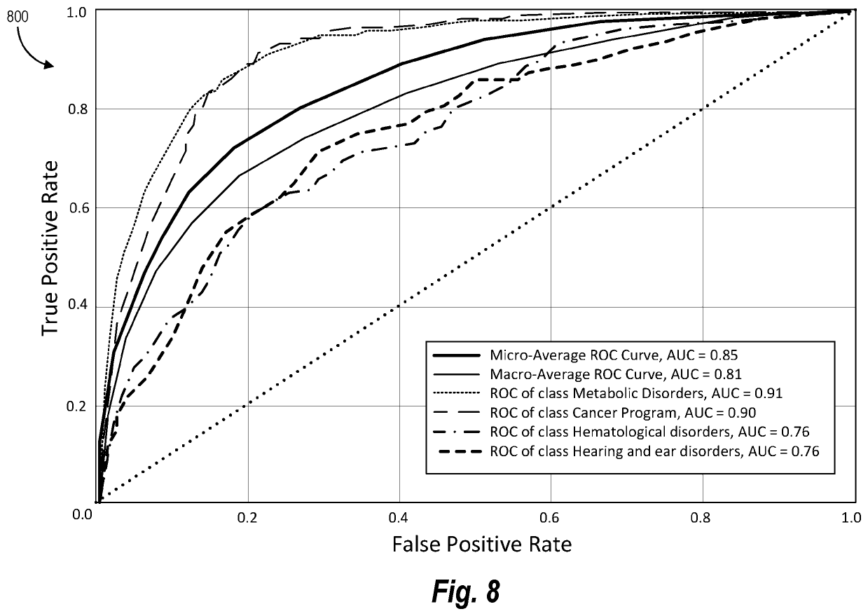

As mentioned, the phenotype prediction system 106 provides accurate predictions of relationships between genes and phenotypes. Indeed, experimenters have demonstrated the accuracy of the phenotype prediction system 106 in generating gene-phenotype relationship predictions utilizing a gene embedding neural network. FIG. 8 illustrates an example table of experimental results from testing the phenotype prediction system 106 in accordance with one or more embodiments.

As illustrated in FIG. 8, the table 800 depicts receiver operation characteristic (ROC) curves for a number of different experiments performed using the phenotype prediction system 106. Specifically, experimenters tested the phenotype prediction system 106 in predicting disease classes from gene embeddings utilizing approximately four thousand GEL panel genes, where 80% of the data was used for training the gene embedding neural network, and 20% of the data was used for testing predictive accuracy. As shown, the phenotype prediction system 106 produces promising ROC curves, such as the ROC curve for the phenotype class of metabolic disorders, with an area under the curve (AOC) of 0.91. Additional results are illustrated in the table. Further experiments were also performed with positive results, but only a subset are illustrated in FIG. 8 for clarity and interpretability.

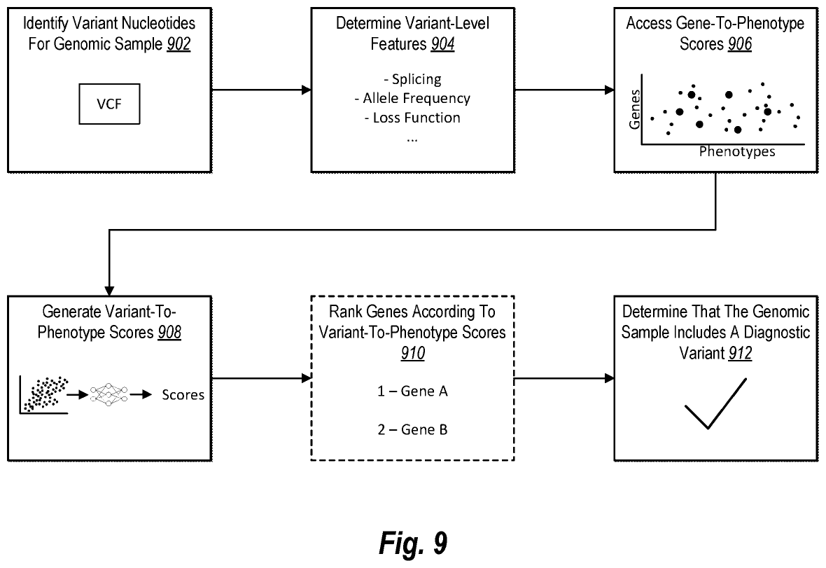

As mentioned above, in certain described embodiments, the phenotype prediction system 106 determines whether a genomic sample of an organism includes a diagnostic variant. In particular, the phenotype prediction system 106 utilizes a diagnostic variant model to process gene-to-phenotype scores (as generated via a gene embedding neural network) and variant-level features for variants of a genomic sample to predict whether one or more of the variants are diagnostic. FIG. 9 illustrates an example overview of determining whether a genomic sample includes a diagnostic variant in accordance with one or more embodiments.

As illustrated in FIG. 9, the phenotype prediction system 106 performs an act 902 to identify variant nucleotides for a genomic sample. To elaborate, the phenotype prediction system 106 accesses, receives, determines, or identifies variant nucleotides for a subset of genes of a sample of an organism, such as a human. In some cases, the phenotype prediction system 106 receives or accesses a variant call file that includes or indicates a genomic sequence for a genomic sample. For instance, the phenotype prediction system 106 receives a variant call file that indicates nucleotide variants at various genomic coordinates among the nucleotides of a genomic sequence.

As also illustrated in FIG. 9, the phenotype prediction system 106 performs an act 904 to determine variant-level features for the variant nucleotides of the genomic sample. In particular, the phenotype prediction system 106 determines features associated with the variant nucleotides identified for the genomic sample. For example, the phenotype prediction system 106 determines variant-level features, such as a pathogenicity score for variant proteins corresponding to variant nucleotides, a splice-site score for the variant nucleotides, an allele frequency corresponding to the variant nucleotides, a genotype for genes of a genomic sample, a loss-of-function status indicating that variant proteins reduce or destroy protein function, or a de novo status of the variant nucleotides as private variants. In some cases, the phenotype prediction system 106 determines variant-level features from external models or external data sources (e.g., other genomic analysis models associated with the sequencing system 114 or associated with third-party systems).

As further illustrated in FIG. 9, the phenotype prediction system 106 performs an act 906 to access gene-to-phenotype scores. For example, the phenotype prediction system 106 accesses, from a lookup table or a database, gene-to-phenotype scores determined using a gene embedding neural network. In some cases, the phenotype prediction system 106 utilizes or implements a gene embedding neural network to determine or generate the gene-to-phenotype scores based on gene embeddings and phenotype labels. Accordingly, the phenotype prediction system 106 determines relationships between the set of genes of a genomic sample and various phenotype labels.

Additionally, the phenotype prediction system 106 performs an act 908 to generate variant-to-phenotype scores. More specifically, the phenotype prediction system 106 utilizes a diagnostic variant model to generate variant-to-phenotype scores that indicate relationships between the variant nucleotides of a genomic sample and various phenotype labels. To elaborate, the phenotype prediction system 106 utilizes a diagnostic variant model in the form of a logistic regression model (or a different type of machine learning model) to determine or predict variant-to-phenotype scores from gene-to-phenotype scores and variant-level features. For instance, the phenotype prediction system 106 determines variant-to-phenotype scores that indicate probabilities of the nucleotide variants of a sample being associated with (or affecting the expression of) various phenotypes, such as rare diseases. Accordingly, the phenotype prediction system 106 can determine relationships between a human patient's variants (as sequenced and stored in a variant call file) and the patient's observed or determined phenotypes (e.g., to determine probabilities of which genes are affecting the observed phenotypes).

Further, in one or more embodiments, the phenotype prediction system 106 performs an act 910 to rank genes according to variant-to-phenotype scores. To elaborate, the phenotype prediction system 106 ranks a subset of genes of a genomic sample according to variant-to-phenotype scores. For example, the phenotype prediction system 106 ranks genes (or nucleotide variants) on a phenotype-by-phenotype basis such that, for a given phenotype, the phenotype prediction system 106 generates a ranked list of genes according to their respective scores of being associated with (or impacting) the given phenotype. In some cases, the phenotype prediction system 106 ranks the genes to identify genes with variant-to-phenotype scores that satisfy a threshold score and/or to identify a threshold number of highest-scoring genes (e.g., the top one or top three genes associated with a phenotype). As shown, the phenotype prediction system 106 ranks Gene A first and Gene B second for a particular phenotype ranking.

As further shown in FIG. 9, the phenotype prediction system 106 performs an act 912 to determine that the genomic sample includes a diagnostic variant. In particular, the phenotype prediction system 106 determines that one or more of the nucleotide variants of a genomic sample are diagnostic in the sense that they satisfy a threshold variant-to-phenotype score and therefore have at least a threshold likelihood of impacting the expression of an observed phenotype (e.g., a rare disease) for an organism (e.g., a patient). In some cases, however, the phenotype prediction system 106 may determine that a genomic sample does not include a diagnostic variant. For instance, if the phenotype prediction system 106 determines that no variant-to-phenotype scores from a set of genes for the sample satisfy a threshold score, then the phenotype prediction system 106 determines that the nucleotide variants for the sample are not diagnostic.

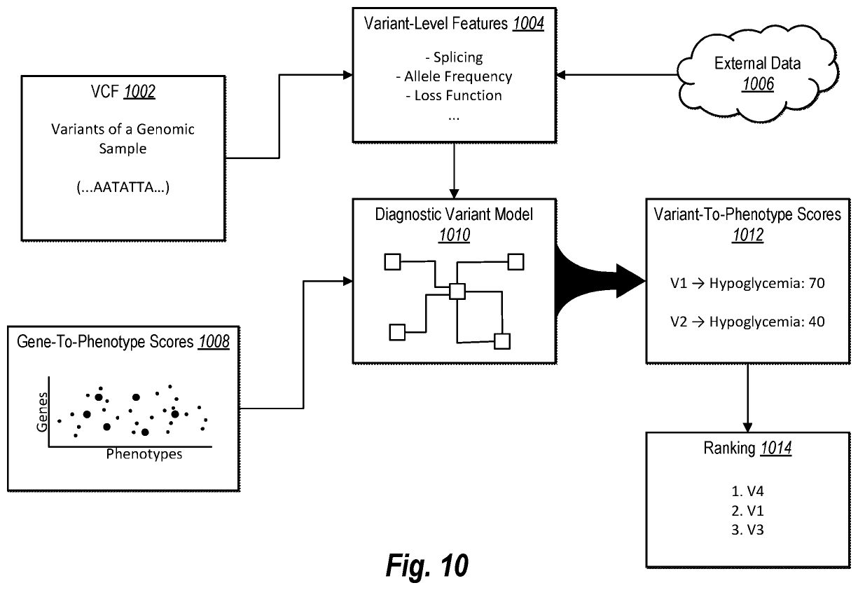

As mentioned, in certain embodiments, the phenotype prediction system 106 generates variant-to-phenotype scores to determine whether a genomic sample includes a diagnostic variant. In particular, the phenotype prediction system 106 utilizes a diagnostic variant model to generate variant-to-phenotype scores for nucleotide variants of a genomic sample. FIG. 10 illustrates an example diagram for generating variant-to-phenotype scores using a diagnostic variant model in accordance with one or more embodiments.

As illustrated in FIG. 10, the phenotype prediction system 106 accesses a variant call file 1002. In particular, the phenotype prediction system 106 accesses, retrieves, or receives the variant call file 1002 that includes indications of nucleotide variants specific to a genomic sample of an organism (e.g., a human patient). In some cases, the variant call file 1002 also indicates other data, such genotypes associated with genes of a genotype sample and/or phenotypes observed in or exhibited by an organism of a genomic sample. In addition, the phenotype prediction system 106 determines variant-level features 1004 for the nucleotide variants indicated by the variant call file 1002. For instance, the phenotype prediction system 106 accesses or receives external data 1006 from one or more genomic analysis models that indicate the variant-level features.

In some cases, the phenotype prediction system 106 receives or accesses data from a PrimateAI model (e.g., PrimateAI-2D or PrimateAI-3D) to determine or access pathogenicity scores for variant proteins corresponding to variant nucleotides. In these or other cases, the phenotype prediction system 106 receives or accesses data from a SpliceAI model to determine or access splice-site scores for variant nucleotides, where, for example, a splice-site score indicates a probability of one or more of the variant nucleotides from the genomic sample as part of a splice site (e.g., a donor site or an acceptor site) or a non-splice site for pre-messenger RNA. Additionally, the phenotype prediction system 106 can access data from a Genome Aggregation Database (gnomAD) to determine or access allele frequencies corresponding to variant nucleotides form the genomic sample. As indicated above, the phenotype prediction system 106 can likewise access and input genotypes for genes from the variant call file 1002 for a particular genomic sample. In some embodiments, the phenotype prediction system 106 can also (or alternatively) access data from other models or databases regarding loss-of-function status indicating whether variant proteins reduce or destroy protein function (or genetic function), or de novo status indicating that variant nucleotides are private variants (e.g., variants not transmitted by or inherited from parents).

As further illustrated in FIG. 10, the phenotype prediction system 106 accesses or determine gene-to-phenotype scores 1008. As described, the phenotype prediction system 106 accesses a lookup table or some other form of data storage that indicates gene-to-phenotype scores for genes of a human genome. In some embodiments, by contrast, the phenotype prediction system 106 executes a gene embedding neural network, as described above, to generate the gene-to-phenotype scores. The phenotype prediction system 106 further utilizes the gene-to-phenotype scores 1008 and the variant-level features to generate variant-to-phenotype scores 1012.

To elaborate, the phenotype prediction system 106 uses the variant-level features 1004 and the gene-to-phenotype scores 1008 as input into a diagnostic variant model 1010. In turn, the diagnostic variant model 1010 processes the variant-level features 1004 and the gene-to-phenotype scores 1008 to determine and encode relationships between the nucleotide variants of the variant call file 1002 and the phenotypes indicated by the gene-to-phenotype scores 1008. For example, the diagnostic variant model 1010 generates probabilities (in the form of variant-to-phenotype scores 1012) that the nucleotide variants are associated with or impact the expression of one or more of the phenotypes. Indeed, based on the encoded relationships between genes and phenotypes (as represented by the gene-to-phenotype scores 1008), the diagnostic variant model 1010 can link nucleotide variants among genes to various (observed) phenotypes as well (e.g., based on the variant level features).

Accordingly, the phenotype prediction system 106 generates variant-to-phenotype scores 1012 that represent the links or relationships between nucleotide variants of a genomic sample and phenotypes observed in the genomic sample. As shown, the phenotype prediction system 106 generates variant-to-phenotype scores 1012 for at least two variants, where the score for variant V1 being associated with hypoglycemia is 70, and the score for variant V2 being associated with hypoglycemia is 40.

As further illustrated in FIG. 10, the phenotype prediction system 106 can generate a ranking 1014 from the variant-to-phenotype scores 1012. More specifically, the phenotype prediction system 106 can generate a phenotype-specific ranking of variant nucleotides. For example, the phenotype prediction system 106 generates a ranking of variants (e.g., all variants of a patient) for a phenotype, such as hypoglycemia (or a phenotype category such as metabolic diseases), by comparing variant-to-phenotype scores associated with the nucleotide variants of a sample. As shown, the phenotype prediction system 106 ranks variant V4 first, variant V1 second, and variant V3 third for a particular phenotype ranking where V4 has the highest variant-to-phenotype score and V3 has the lowest. In some cases, the phenotype prediction system 106 can select a highest-ranked variant (or a variant with at least a threshold variant-to-phenotype score) as a diagnostic variant for a sample of an organism.

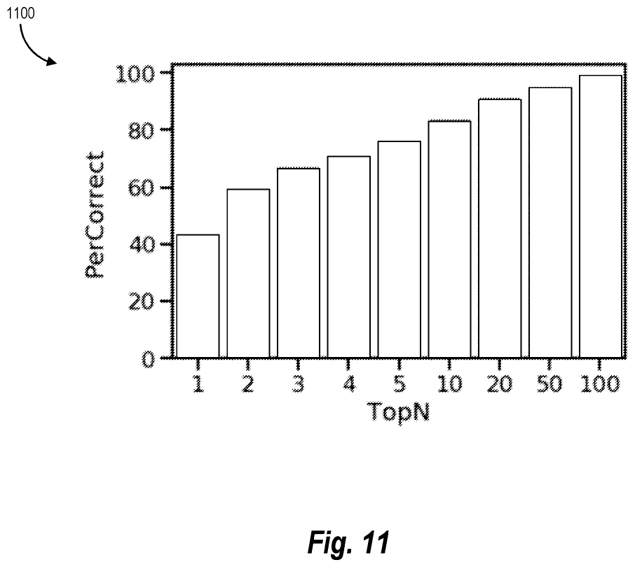

As suggested above, experimenters have demonstrated the accuracy of the phenotype prediction system 106 in generating variant-to-phenotype scores for determining diagnostic variants. In particular, experimenters have evaluated the performance of the phenotype prediction system 106 in identifying nucleotide variants that correspond to particular phenotypes. FIG. 11 illustrates an example table of experimental results of the phenotype prediction system 106 in determining diagnostic variants in accordance with one or more embodiments.

As illustrated in FIG. 11, the table 1100 includes experimental results for the phenotype prediction system 106. In particular, experimenters trained the diagnostic variant model of the phenotype prediction system 106 on variant data (e.g., variant call file data) of approximately 2500 patients with approximately 500 variants per patient. The table 1100 indicates the topN results for the top N variant nucleotides in a ranking, along with a percent correct (“PerCorrect”) of the top N results. As shown in the table 1100, upon testing, in approximately 80% of cases (e.g., for 80% of patients whose genes were tested), the phenotype prediction system 106 determines or identifies a correct diagnostic variant for genes of a sample within the top ten ranked variants (e.g., within a list of the top ten variants with the highest variant-to-phenotype scores for a given phenotype). In 40% of the test cases (e.g., test gene sets from test patients), the phenotype prediction system 106 determined the correct diagnostic variant as the top-ranked nucleotide variant (e.g., the variant with the highest variant-to-phenotype score).

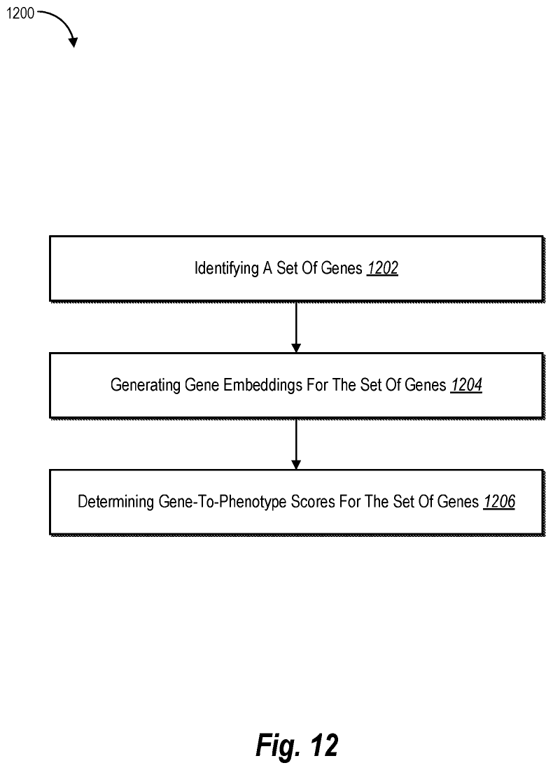

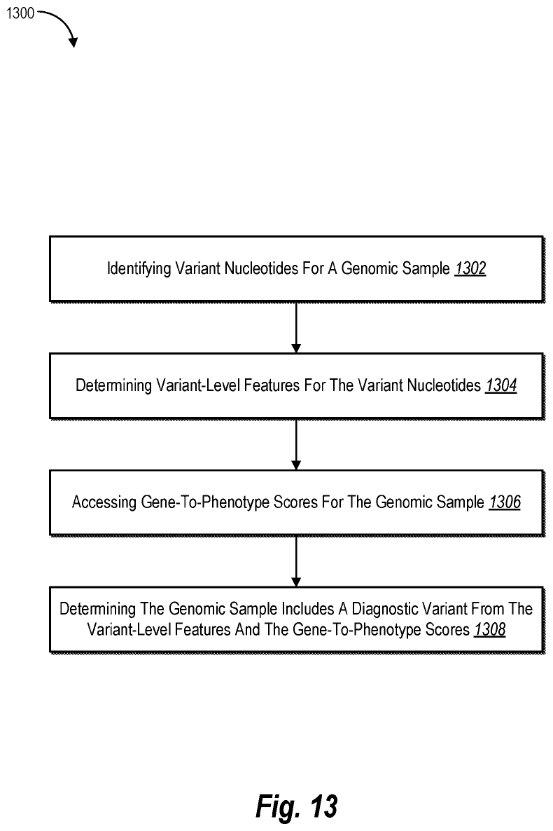

Turning now to FIG. 12, this figure illustrates an example flowchart of a series of acts of determining gene-to-phenotype scores for a set of genes using a gene embedding neural network. Thereafter, FIG. 13, illustrates an example flowchart of a series of acts for determining variant-to-phenotype scores for variants of a sample using a diagnostic variant model. While FIGS. 12-13 illustrates acts according to one or embodiments, alternative embodiments may omit, add to, reorder, and/or modify any of the acts shown in FIGS. 12-13. The acts of FIGS. 12-13 can be performed as part of a method. Alternatively, a non-transitory computer readable storage medium can comprise instructions that, when executed by one or more processors, cause a computing device to perform the acts depicted in FIGS. 12-13. In still further embodiments, a system comprising at least one processor and a non-transitory computer readable medium comprising instructions that, when executed by one or more processors, cause the system to perform the acts of FIGS. 12-13.

As shown in FIG. 12, the series of acts 1200 includes an act 1202 of identifying a set of genes. In addition, the series of acts 1200 includes an act 1204 of generating gene embeddings for the set of genes. As shown, the series of acts 1200 includes an act 1206 of determining gene-to-phenotype scores for a set of genes. In some embodiments, the series of acts 1200 includes acts to perform any of the operations described in the following clauses:

CLAUSE 1. A method comprising:
identifying a set of genes from a human genome;
generating, using a gene embedding neural network comprising parameters learned from one or more gene-to-gene graphs, gene embeddings encoding relationships between the set of genes from the human genome; and
determining, based on the gene embeddings, gene-to-phenotype scores indicating respective probabilities of the set of genes from the human genome being associated with various phenotypes.

CLAUSE 2. The method of clause 1, further comprising generating the gene embeddings by utilizing the gene embedding neural network to generate a latent vector encoding a gene and its relationships to other genes within the one or more gene-to-gene graphs.

CLAUSE 3. The method of any of clauses 1-2, further comprising generating the gene embeddings by encoding the latent vector having a number of dimensions corresponding to a number of neurons in a penultimate layer of the gene embedding neural network.

CLAUSE 4. The method of any of clauses 1-3, further comprising determining the relationships between the set of genes within the one or more gene-to-gene graphs according to distances between the gene embeddings within a latent space of the gene embedding neural network.

CLAUSE 5. The method of any of clauses 1-4, further comprising:
determining the gene-to-phenotype scores by utilizing an output layer of the gene embedding neural network; and
generating, based on the gene embeddings and the gene-to-phenotype scores, a gene-to-phenotype matrix indicating probabilities of the set of genes being associated with a set of labeled phenotypes.

CLAUSE 6. The method of any of clauses 1-5, further comprising clustering, utilizing the gene embedding neural network, the gene embeddings into phenotype clusters within a latent space according to distances relative to one another and according to phenotype labels.

CLAUSE 7. The method of any of clauses 1-6, further comprising training the gene embedding neural network to generate gene-to-phenotype scores utilizing a two-stage training process comprising an unsupervised training stage and a supervised training stage based on gene-to-gene graphs and clinical phenotype labels.

CLAUSE 8. The method of any of clauses 1-6, further comprising training the gene embedding neural network by utilizing an unsupervised training stage based on a graph representation learning algorithm to learn network parameters of the gene embedding neural network for defining a structure of the one or more gene-to-gene graphs.

CLAUSE 9. The method of any of clauses 1-6, further comprising training the gene embedding neural network by further utilizing a supervised fine-tuning stage to modify the network parameters learned during the unsupervised training stage for determining probabilities of gene embeddings corresponding to clinically determined phenotype labels.

CLAUSE 10. The method of any of clauses 1-9, wherein the one or more gene-to-gene graphs comprise one or more of a protein-protein interaction (PPI) graph, a gene coexpression (CE) graph, gene-to-gene-sequence similarity network, or a biological pathway graph representing genes that share common biological pathways.

CLAUSE 11. The method of any of clauses 1-10, wherein identifying the set of genes from the human genome comprises identifying one or more genes from the human genome without corresponding phenotype labels.

CLAUSE 12. The method of any of clauses 1-11, further comprising expanding a gene panel comprising a sequence of listed genes corresponding to one or more variants by determining an additional gene to add to the gene panel based on gene-to-phenotype scores.

CLAUSE 13. The method of any of clauses 1-12, further comprising determining, utilizing a diagnostic variant model and based on the gene-to-phenotype scores, that a genomic sample comprises a diagnostic variant associated with a phenotype of the various phenotypes.

As mentioned, FIG. 13 illustrates a series of acts 1300 for determining variant-to-phenotype scores in accordance with one or more embodiments. As illustrated in FIG. 13, the series of acts 1300 includes an act 1302 of identifying variant nucleotides for a genomic sample. In addition, the series of acts 1300 includes an act 1304 of determining variant-level features for the variant nucleotides. As shown, the series of acts 1300 also includes an act 1306 of accessing gene-to-phenotype scores for the genomic sample. Further, the series of acts 1300 includes an act 1308 of determining the genomic sample includes a diagnostic variant from the variant-level features and the gene-to-phenotype scores. In some embodiments, the series of acts 1300 includes acts to perform any of the operations described in the following clauses:

CLAUSE 14. A method comprising:
identifying variant nucleotides for a subset of genes of a genomic sample;
determining variant-level features associated with the variant nucleotides for the subset of genes of the genomic sample;
accessing, for a set of genes from a genome associated with the genomic sample and processed by a gene embedding neural network, gene-to-phenotype scores indicating respective probabilities of the set of genes being associated with phenotypes determined for an organism of the genomic sample; and
determining, utilizing a diagnostic variant model and based on the variant-level features and the gene-to-phenotype scores, the genomic sample comprises a diagnostic variant that is associated with a phenotype of the phenotypes determined for the organism.

CLAUSE 15. The method of clause 14, wherein the variant-level features comprise one or more of pathogenicity scores indicating a degree to which variant proteins corresponding to the variant nucleotides are benign or pathogenic, splice-site scores indicating a probability of one or more of the variant nucleotides being part of a splice site or a non-splice site for pre-messenger RNA, allele frequencies corresponding to the variant nucleotides, genotypes for the subset of genes of the genomic sample, loss-of-function statuses indicating that the variant proteins reduce or destroy protein function, or de novo status of the variant nucleotides as private variants.

CLAUSE 16. The method of any of clauses 14-15, further comprising generating the gene-to-phenotype scores by:
generating, utilizing the gene embedding neural network, gene embeddings encoding relationships between the set of genes from the genome associated with the genomic sample; and determining, from the gene embeddings, probabilities of variants within the set of genes affecting expression of one or more of a set of clinically determined phenotypes for the genomic sample.

CLAUSE 17. The method of any of clauses 14-16, further comprising determining the genomic sample comprises the diagnostic variant by generating, utilizing the diagnostic variant model to process the variant-level features and the gene-to-phenotype scores, variant-to-phenotype scores indicating probabilities of the variant nucleotides for the subset of genes affecting expression of the phenotypes determined for the organism.

CLAUSE 18. The method of clause 17, further comprising determining the genomic sample comprises the diagnostic variant by further:
ranking the subset of genes comprising the variant nucleotides associated with the genomic sample according to the variant-to-phenotype scores; and
selecting, from the subset of genes, a gene comprising a variant nucleotide with a highest variant-to-phenotype score from the ranking.

CLAUSE 19. The method of any of clauses 14-18, wherein the diagnostic variant model comprises a logistic regression model, a linear regression model, a random forest, a decision tree, or a neural network.

CLAUSE 20. The method of any of clauses 14-19, wherein the subset of genes of the genomic sample comprises genes reflecting variant nucleotides.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using y-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996), “Real-time DNA sequencing using detection of pyrophosphate release,” Analytical Biochemistry 242(1), 84-9; Ronaghi, M. (2001), “Pyrosequencing sheds light on DNA sequencing,” Genome Res. 11(1), 3-11; Ronaghi, M., Uhlen, M. and Nyren, P. (1998), “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO Ser. No. 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO Ser. No. 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing,” Trends Biotechnol. 18, 147-151 (2000); Deamer, D. and D. Branton, “Characterization of nucleic acids by nanopore analysis.” Acc. Chem. Res. 35:817-825 (2002); Li, J., M. Gershow, D. Stein, E. Brandin, and J. A. Golovchenko, “DNA molecules and configurations in a solid-state nanopore microscope,” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores,” Clin. Chem. 53, 1996-2001 (2007); Healy, K., “Nanopore-based single-molecule DNA analysis,” Nanomed. 2, 459-481 (2007); Cockroft, S. L., Chu, J., Amorin, M. & Ghadiri, M. R., “A single-molecule nanopore device detects DNA polymerase activity with single-nucleotide resolution,” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and y-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al., “Zero-mode waveguides for single-molecule analysis at high concentrations,” Science 299, 682-686 (2003); Lundquist, P. M. et al., “Parallel confocal detection of single molecules in real time,” Opt. Lett. 33, 1026-1028 (2008); Korlach, J. et al., “Selective aluminum passivation for targeted immobilization of single DNA polymerase molecules in zero-mode waveguide nano structures,” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. 2009/0026082 A1; U.S. 2009/0127589 A1; U.S. 2010/0137143A1; or U.S. 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm^{2, 10,000} features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in U.S. 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the phenotype prediction system 106 can include software, hardware, or both. For example, the components of the phenotype prediction system 106 can include one or more instructions stored on a computer-readable storage medium and executable by processors of one or more computing devices (e.g., the client device 108). When executed by the one or more processors, the computer-executable instructions of the phenotype prediction system 106 can cause the computing devices to perform the bubble detection methods described herein. Alternatively, the components of the phenotype prediction system 106 can comprise hardware, such as special purpose processing devices to perform a certain function or group of functions. Additionally, or alternatively, the components of the phenotype prediction system 106 can include a combination of computer-executable instructions and hardware.

Furthermore, the components of the phenotype prediction system 106 performing the functions described herein with respect to the phenotype prediction system 106 may, for example, be implemented as part of a stand-alone application, as a module of an application, as a plug-in for applications, as a library function or functions that may be called by other applications, and/or as a cloud-computing model. Thus, components of the phenotype prediction system 106 may be implemented as part of a stand-alone application on a personal computing device or a mobile device. Additionally, or alternatively, the components of the phenotype prediction system 106 may be implemented in any application that provides sequencing services including, but not limited to Illumina PrimateAI, PrimateAI-2D, PrimateAI-3D, or SpliceAI software. “Illumina,” “PrimateAI,” and “SpliceAI,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (Saas), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 14 illustrates a block diagram of a computing device 1400 (e.g., the client device 108, the server device(s) 102, and/or the server device(s) 104) that may be configured to perform one or more of the processes described above. One will appreciate that one or more computing devices such as the computing device 1400 may implement the phenotype prediction system 106 and the sequencing system 114. As shown by FIG. 14, the computing device 1400 can comprise a processor 1402, a memory 1404, a storage device 1406, an I/O interface 1408, and a communication interface 1410, which may be communicatively coupled by way of a communication infrastructure 1414. In certain embodiments, the computing device 1400 can include fewer or more components than those shown in FIG. 14. The following paragraphs describe components of the computing device 1400 shown in FIG. 14 in additional detail.

In one or more embodiments, the processor 1402 includes hardware for executing instructions, such as those making up a computer program. As an example, and not by way of limitation, to execute instructions for dynamically modifying workflows, the processor 1402 may retrieve (or fetch) the instructions from an internal register, an internal cache, the memory 1404, or the storage device 1406 and decode and execute them. The memory 1404 may be a volatile or non-volatile memory used for storing data, metadata, and programs for execution by the processor(s). The storage device 1406 includes storage, such as a hard disk, flash disk drive, or other digital storage device, for storing data or instructions for performing the methods described herein.

The I/O interface 1408 allows a user to provide input to, receive output from, and otherwise transfer data to and receive data from computing device 1400. The I/O interface 1408 may include a mouse, a keypad or a keyboard, a touch screen, a camera, an optical scanner, network interface, modem, other known I/O devices or a combination of such I/O interfaces. The I/O interface 1408 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1408 is configured to provide graphical data to a display for presentation to a user. The graphical data may be representative of one or more graphical user interfaces and/or any other graphical content as may serve a particular implementation.

The communication interface 1410 can include hardware, software, or both. In any event, the communication interface 1410 can provide one or more interfaces for communication (such as, for example, packet-based communication) between the computing device 1400 and one or more other computing devices or networks. As an example, and not by way of limitation, the communication interface 1410 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1410 may facilitate communications with various types of wired or wireless networks. The communication interface 1410 may also facilitate communications using various communication protocols. The communication infrastructure 1414 may also include hardware, software, or both that couples components of the computing device 1400 to each other. For example, the communication interface 1410 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium comprising instructions that, when executed by the at least one processor, cause the system to:
identify a set of genes from a human genome;
generate, using a gene embedding neural network comprising parameters learned from one or more gene-to-gene graphs, gene embeddings encoding relationships between the set of genes from the human genome; and
determine, based on the gene embeddings, gene-to-phenotype scores indicating respective probabilities of the set of genes from the human genome being associated with various phenotypes.

2. The system of claim 1, further comprising instructions that, when executed by the at least one processor, cause the system to generate the gene embeddings by utilizing the gene embedding neural network to generate a latent vector encoding a gene and its relationships to other genes within the one or more gene-to-gene graphs.

3. The system of claim 1, further comprising instructions that, when executed by the at least one processor, cause the system to generate the gene embeddings by encoding a latent vector having a number of dimensions corresponding to a number of neurons in a penultimate layer of the gene embedding neural network.

4. The system of claim 1, further comprising instructions that, when executed by the at least one processor, cause the system to determine the relationships between the set of genes within the one or more gene-to-gene graphs according to distances between the gene embeddings within a latent space of the gene embedding neural network.

5. The system of claim 1, further comprising instructions that, when executed by the at least one processor, cause the system to:
determine the gene-to-phenotype scores by utilizing an output layer of the gene embedding neural network; and
generate, based on the gene embeddings and the gene-to-phenotype scores, a gene-to-phenotype matrix indicating probabilities of the set of genes being associated with a set of labeled phenotypes.

6. The system of claim 1, further comprising instructions that, when executed by the at least one processor, cause the system to cluster, utilizing the gene embedding neural network, the gene embeddings into phenotype clusters within a latent space according to distances relative to one another and according to phenotype labels.

7. The system of claim 1, further comprising instructions that, when executed by the at least one processor, cause the system to train the gene embedding neural network to generate gene-to-phenotype scores utilizing a two-stage training process comprising an unsupervised training stage and a supervised training stage based on gene-to-gene graphs and clinical phenotype labels.

8. The system of claim 7, further comprising instructions that, when executed by the at least one processor, cause the system to train the gene embedding neural network by utilizing an unsupervised training stage based on a graph representation learning algorithm to learn network parameters of the gene embedding neural network for defining a structure of the one or more gene-to-gene graphs.

9. The system of claim 8, further comprising instructions that, when executed by the at least one processor, cause the system to train the gene embedding neural network by further utilizing a supervised fine-tuning stage to modify the network parameters learned during the unsupervised training stage for determining probabilities of gene embeddings corresponding to clinically determined phenotype labels.

10. The system of claim 1, wherein the one or more gene-to-gene graphs comprise one or more of a protein-protein interaction (PPI) graph, a gene coexpression (CE) graph, gene-to-gene-sequence similarity network, or a biological pathway graph representing genes that share common biological pathways.

11. The system of claim 1, wherein the instructions, when executed by the at least one processor, cause the system to identify the set of genes from the human genome by identifying one or more genes from the human genome without corresponding phenotype labels.

12. The system of claim 1, further comprising instructions that, when executed by the at least one processor, cause the system to expand a gene panel comprising a sequence of listed genes corresponding to one or more variants by determining an additional gene to add to the gene panel based on gene-to-phenotype scores.

13. The system of claim 1, further comprising instructions that, when executed by the at least one processor, cause the system to determine, utilizing a diagnostic variant model and based on the gene-to-phenotype scores, that a genomic sample comprises a diagnostic variant associated with a phenotype of the various phenotypes.

14. A system comprising:
at least one processor; and
a non-transitory computer readable medium comprising instructions that, when executed by the at least one processor, cause the system to:
identify variant nucleotides for a subset of genes of a genomic sample;
determine variant-level features associated with the variant nucleotides for the subset of genes of the genomic sample;
access, for a set of genes from a genome associated with the genomic sample and processed by a gene embedding neural network, gene-to-phenotype scores indicating respective probabilities of the set of genes being associated with phenotypes determined for an organism of the genomic sample; and
determine, utilizing a diagnostic variant model and based on the variant-level features and the gene-to-phenotype scores, the genomic sample comprises a diagnostic variant that is associated with a phenotype of the phenotypes determined for the organism.

15. The system of claim 14, wherein the variant-level features comprise one or more of pathogenicity scores indicating a degree to which variant proteins corresponding to the variant nucleotides are benign or pathogenic, splice-site scores indicating a probability of one or more of the variant nucleotides being part of a splice site or a non-splice site for pre-messenger RNA, allele frequencies corresponding to the variant nucleotides, genotypes for the subset of genes of the genomic sample, loss-of-function statuses indicating that the variant proteins reduce or destroy protein function, or de novo status of the variant nucleotides as private variants.

16. The system of claim 14, further comprising instructions that, when executed by the at least one processor, cause the system to generate the gene-to-phenotype scores by:
generating, utilizing the gene embedding neural network, gene embeddings encoding relationships between the set of genes from the genome associated with the genomic sample; and
determining, from the gene embeddings, probabilities of variants within the set of genes affecting expression of one or more of a set of clinically determined phenotypes for the genomic sample.

17. The system of claim 14, further comprising instructions that, when executed by the at least one processor, cause the system to determine the genomic sample comprises the diagnostic variant by generating, utilizing the diagnostic variant model to process the variant-level features and the gene-to-phenotype scores, variant-to-phenotype scores indicating probabilities of the variant nucleotides for the subset of genes affecting expression of the phenotypes determined for the organism.

18. The system of claim 17, further comprising instructions that, when executed by the at least one processor, cause the system to determine the genomic sample comprises the diagnostic variant by further:
ranking the subset of genes comprising the variant nucleotides associated with the genomic sample according to the variant-to-phenotype scores; and
selecting, from the subset of genes, a gene comprising a variant nucleotide with a highest variant-to-phenotype score from the ranking.

19. The system of claim 14, wherein the diagnostic variant model comprises a logistic regression model, a linear regression model, a random forest, a decision tree, or a neural network.

20. The system of claim 14, wherein the subset of genes of the genomic sample comprises genes reflecting variant nucleotides.
